(19) United States
(12) Patent Application Publication
(10) Pub. No.: US 2026/0360535 A1
(43) Pub. Date: Nov. 12, 2026

(54) SYSTEMS AND METHODS FOR TRACKING PERSONALIZED METHYLATION BIOMARKERS FOR THE DETECTION OF DISEASE

(71) Applicant: Foundation Medicine, Inc., Boston, MA (US)

(72) Inventors: Nicole LAMBERT, San Diego, CA (US); Alexander De Jong ROBERTSON, Princeton, NJ (US); Ashley LINARES, Carlsbad, CA (US); Yexun WANG, San Diego, CA (US); Neil PETERMAN, San Francisco, CA (US)

(73) Assignee: Foundation Medicine, Inc., Boston, MA (US)

(21) Appl. No.: 19/131,851

(22) PCT Filed: Nov. 22, 2023
(86) PCT No.: PCT/US2023/080922

§ 371 Date: May 21, 2025

Related U.S. Application Data
(60) Provisional application No. 63/427,695, filed on Nov. 23, 2022.

(51) Int. Cl. G16H 50/20; C12Q 1/6809; C12Q 1/6855; C12Q 1/6874; G16B 20/20; G16B 30/10; G16H 50/70

(52) U.S. Cl. CPC G16H 50/20; C12Q 1/6809; C12Q 1/6855; C12Q 1/6874; G16B 20/20; G16B 30/10; G16H 50/70

(57) ABSTRACT

Methods for generating personalized methylation biomarkers and using them for the detection of disease are described. The methods may comprise, for example, receiving a first set of sequence read data derived from a first sample from a subject, determining a methylation status for each of a plurality of genomic loci based on sequence read data, identifying a subset of the genomic loci that are differentially methylated in the first sample based on a comparison of their methylation status to at least one predetermined methylation status threshold, receiving a second set of sequence read data derived from a liquid biopsy sample from the subject, determining a methylation status for the subset of differentially methylated genomic loci based on the second set of sequence read data, and detecting a disease signal based on methylation status determined for the subset of differentially methylated genomic loci in the second set of sequence read data.

### CROSS-REFERENCE TO RELATED APPLICATIONS

This application claims the priority benefit of U.S. Provisional Patent Application Ser. No. 63/427,695, filed Nov. 23, 2022, the contents of which are incorporated herein by reference in their entirety.

### FIELD OF THE DISCLOSURE

The present disclosure relates generally to methods and systems for the detection of disease, and more specifically, to methods and systems for tracking personalized methylation biomarkers for the detection of disease.

### BACKGROUND

Accurate and sensitive detection of circulating tumor DNA (ctDNA) provides an important indicator of the presence of minimum residual disease (MRD). The detection of ctDNA provides clinically-actionable patient information, enables a more accurate estimate of recurrence risk, and has the potential to alter treatment/monitoring paradigms. ctDNA, however, is typically found in low levels, and is difficult to detect due to technical limitations such as high error rates in library preparation and sequencing methods. Clonal hematopoiesis of indeterminate potential (CHiP), the accumulation of somatic mutations in blood cell progenitors, are often indistinguishable from cancer mutations found in plasma and thus may also confound the detection of ctDNA. Improved methods are needed for analyzing ctDNA for the assessment of disease risk. The present disclosure addresses these needs.

### BRIEF SUMMARY OF THE INVENTION

Disclosed herein are methods and systems for tracking personalized methylation biomarkers for the detection of disease. Personalized methylation biomarkers can be identified in DNA extracted from patient tissue samples, and subsequently tracked in cell-free DNA (cfDNA) extracted from matched patient plasma samples. Existing methods that analyze cfDNA in order to detect ctDNA often suffer from lack of detection sensitivity and/or interference from CHIP, as noted above. By first characterizing a set of personalized methylation biomarkers (e.g., a set of differentially methylated variants) in high signal tumor tissue, the disclosed methods are less prone to specificity errors and may improve ctDNA detection limits. The disclosed methods are also less susceptible to CHIP interference because, in general, methylation of CHIP variants is far less common than methylation of ctDNA, and may be less common than methylation of ctDNA in certain targeted regions. Furthermore, the methods and systems described herein benefit from the advantages generally seen in liquid biopsy-based, noninvasive cfDNA analysis and ctDNA detection schemes.

Disclosed herein are methods comprising: providing a plurality of nucleic acid molecules obtained from a first sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a first set of sequence read data for a plurality of sequence reads derived from the first sample that represent the captured nucleic acid molecules; receiving, at one or more processors, the first set of sequence read data for the plurality of sequence reads derived from the first sample from the subject; determining, using the one or more processors, a methylation status for each of a plurality of genomic loci based on the first set of sequence read data; and identifying, using the one or more processors, a subset of the plurality of genomic loci that are differentially methylated in the first sample based on a comparison of their methylation status to one or more predetermined methylation status thresholds.

In some embodiments, the subset of differentially methylated genomic loci identified for the subject are used to monitor a disease status of the subject, the method further comprising: receiving, at the one or more processors, a second set of sequence read data for a plurality of sequence reads derived from a second sample from the subject; determining, using the one or more processors, a methylation status for the subset of differentially methylated genomic loci based on the second set of sequence read data; and detecting a disease signal in the subject based on the methylation status determined for the subset of differentially methylated genomic loci in the second set of sequence read data, wherein the disease signal is indicative of the disease status of the subject.

In some embodiments, the method further comprises estimating a disease-associated DNA fraction for the second sample based on the methylation status determined at the subset of differentially methylated genomic loci in the second set of sequence read data. In some embodiments, the estimation of disease-associated DNA fraction is based on a fraction of the subset of differentially methylated genomic loci for which methylation is detected, an average level of methylation for the subset of differentially methylated genomic loci, a fraction of differentially methylated sequence reads in the second set of sequence read data, or an absolute number of differentially methylated reads in the second set of sequence read data. In some embodiments, the plurality of genomic loci ‘are identified based on an analysis of methylation status at genomic loci in one or more samples from healthy individuals.

In some embodiments, the method further comprises: receiving, at the one or more processors, a third set of sequence read data for a plurality of sequence reads derived from a disease-free control sample from the subject; determining, using the one or more processors, a methylation status for each of the plurality of genomic loci based on the third set of sequence read data; and excluding genomic loci from the subset of differentially methylated genomic loci if their methylation status is the same for the first sample and the disease-free control sample.

In some embodiments, the first sample is a tumor tissue sample. In some embodiments, each set of sequence read data comprises sequence read data derived from a methylation sequencing method. In some embodiments, the methylation sequencing method comprises use of a bisulfite conversion reaction or an enzymatic conversion reaction to convert non-methylated cytosine to uracil. In some embodiments, the methylation sequencing method comprises use of a chemical or enzymatic conversion reaction to convert methylated cytosine to uracil.

In some embodiments, the subject is suspected of having or is determined to have cancer. In some embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the method further comprises treating the subject with an anti-cancer therapy. In some embodiments, the anti-cancer therapy comprises a targeted anti-cancer therapy. In some embodiments, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some embodiments, the method further comprises obtaining the first and/or second sample from the subject. In some embodiments, the first and/or second sample comprises a tissue biopsy sample, a liquid biopsy sample, or a normal control. In some embodiments, the first and/or second sample is a liquid biopsy sample and comprises blood, plasma, cerebrospinal fluid, sputum, stool, urine, or saliva. In some embodiments, the first and/or second sample is a liquid biopsy sample and comprises circulating tumor cells (CTCs). In some embodiments, the first and/or second sample is a liquid biopsy sample and comprises cell-free DNA (cfDNA), circulating tumor DNA (ctDNA), or any combination thereof.

In some embodiments, the plurality of nucleic acid molecules comprises a mixture of tumor nucleic acid molecules and non-tumor nucleic acid molecules. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the first and/or second sample comprises a liquid biopsy sample, and the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some embodiments, the one or more adapters comprise amplification primers, flow cell adaptor sequences, substrate adapter sequences, or sample index sequences. In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule.

In some embodiments, the amplifying nucleic acid molecules comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or Sanger sequencing technique. In some embodiments, the sequencing comprises massively parallel sequencing, and the massively parallel sequencing technique comprises next generation sequencing (NGS). In some embodiments, the sequencer comprises a next generation sequencer.

In some embodiments, one or more of the plurality of sequencing reads overlap one or more gene loci within one or more subgenomic intervals in the sample. In some embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In some embodiments, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, ZNF703, or any combination thereof.

In some embodiments, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In some embodiments, the method further comprises: generating, by the one or more processors, a report indicating the presence or absence of detected disease in the subject; and transmitting the report to a healthcare provider. In some embodiments, the report is transmitted via a computer network or a peer-to-peer connection.

Disclosed herein are methods for identifying a subset of differentially methylated genomic loci in a subject, the method comprising: a) receiving, at one or more processors, a first set of sequence read data for a plurality of sequence reads derived from a first sample from the subject; b) determining, using the one or more processors, a methylation status for each of a plurality of genomic loci based on the first set of sequence read data; and c) identifying, using the one or more processors, a subset of the plurality of genomic loci that are differentially methylated in the first sample based on a comparison of their methylation status to one or more predetermined methylation status thresholds.

In some embodiments, the subset of differentially methylated genomic loci identified for the subject are used to monitor a disease status of the subject, the method further comprising: d) receiving, at one or more processors, a second set of sequence read data for a plurality of sequence reads derived from a liquid biopsy sample from the subject; e) determining, using the one or more processors, a methylation status for the subset of differentially methylated genomic loci based on the second set of sequence read data; and f) detecting a disease signal in the subject based on the methylation status determined for the subset of differentially methylated genomic loci in the second set of sequence read data, wherein the disease signal is indicative of the disease status of the subject.

In some embodiments, the method further comprises estimating a disease-associated DNA fraction for the liquid biopsy sample based on the methylation status determined at the subset of differentially methylated genomic loci in the second set of sequence read data. In some embodiments, the estimation of disease-associated DNA fraction is based on a fraction of the subset of differentially methylated genomic loci for which methylation is detected, an average level of methylation for the subset of differentially methylated genomic loci, a fraction of differentially methylated sequence reads in the second set of sequence read data, or an absolute number of differentially methylated reads in the second set of sequence read data.

In some embodiments, the plurality of genomic loci are identified based on an analysis of methylation status at genomic loci in one or more samples from healthy individuals. In some embodiments, the method further comprises: receiving, at the one or more processors, a third set of sequence read data for a plurality of sequence reads derived from a disease-free control sample from the subject; determining, using the one or more processors, a methylation status for each of the plurality of genomic loci based on the third set of sequence read data; and excluding genomic loci from the subset of differentially methylated genomic loci identified in the above methods, if their methylation status is the same for the first sample and the disease-free control sample.

In some embodiments, the disease-free control sample comprises a matched normal tissue sample from the subject. In some embodiments, the disease-free control sample comprises a buffy coat sample from the subject. In some embodiments, the disease-free control sample comprises an adjacent normal tissue sample from the subject. In some embodiments, the detection of the disease signal is further based on detection of one or more somatic single base substitutions, indels, or structural variants based on the second set of sequence read data.

In some embodiments, the plurality of genomic loci comprises at least 10, at least 100, at least 1,000, at least 5,000, at least 10,000, at least 20,000, at least 30,000, at least 40,000, at least 50,000, at least 60,000, at least 70,000, at least 80,000, at least 90,000, at least 100,000, at least 120,000, at last 140,000, at least 160,000, at least 180,000, or at least 200,000 genomic loci.

In some embodiments, the identified subset of the plurality of genomic loci is tumor-specific for a subject that has been diagnosed with cancer. In some embodiments, the identified subset of the plurality of genomic loci is treatment-specific for a subject that has previously been treated with an anti-cancer therapy. In some embodiments, the identified subset of the plurality of genomic loci is patient-specific. In some embodiments, the one or more predetermined methylation status thresholds are determined based on an average degree of methylation detected at a specified plurality of genomic loci in a cohort of healthy and diseased subjects.

In some embodiments, the one or more predetermined methylation status thresholds are determined based on an analysis of cluster consensus methylation fractions (CCMFs) for a cohort of healthy and diseased subjects. In some embodiments, the one or more predetermined methylation status thresholds are determined based on an analysis of cluster consensus unmethylation fractions (CCUFs) for a cohort of healthy and diseased subjects.

In some embodiments, determining a cluster consensus methylation fraction (CCMF) or a cluster consensus unmethylation fraction (CCUF) for each of the subjects in the cohort of heathy and diseased subjects comprises: receiving, at one or more processors, sequence read data for a plurality of sequence reads derived from a sample from a subject, wherein at least a portion of the plurality of sequence reads corresponds to a genomic locus comprising a cluster of two or more CpG dinucleotides; determining, using the one or more processors, a consensus methylation pattern or a consensus unmethylation pattern for the cluster, wherein the consensus methylation pattern represents each CpG dinucleotide in the cluster for which methylation was detected based on the absence cytosine conversion in at least one sequence read from the sequence read data, and wherein the consensus unmethylation pattern represents each CpG dinucleotide in the cluster for which methylation was not detected; generating, using the one or more processors, a cluster consensus fraction (CCF) for the cluster, wherein the CCF represents a fraction of sequence reads corresponding to the cluster that show the consensus methylation pattern, or a fraction of sequence reads corresponding to the cluster that show the consensus unmethylation pattern, out of a total number of sequence reads from the plurality corresponding to the cluster; and detecting one or more of the methylation level or the unmethylation level of the cluster based on the CCF.

In some embodiments, the one or more predetermined methylation status thresholds are determined for the subject based on a comparison of the methylation status for each of the plurality of genomic loci as determined based on the first set of sequence read data and as determined based on a set of sequence read data derived from a control sample from the subject.

In some embodiments, the control sample comprises a matched normal tissue sample from the subject. In some embodiments, the methylation status of a given genomic locus of the plurality of genomic loci comprises a hypermethylation status, a hypomethylation status, or a hemimethylation status. In some embodiments, the liquid biopsy sample comprises blood, plasma, serum, cerebrospinal fluid, sputum, stool, urine, or saliva.

In some embodiments, the method further comprises performing a sequencing assay to generate the first set of sequence read data, the second set of sequence read data, or both. In some embodiments, the method further comprises performing a sequencing assay to generate the third set of sequence read data. In some embodiments, the sequencing assay used to generate the first set, second set, or third set of sequence read data comprises a targeted sequencing method. In some embodiments, the targeted sequencing method comprises use of a set of bait molecules designed to target the subset of differentially methylated genomic loci.

In some embodiments, the set of bait molecules is customized to target a set of differentially methylated genomic loci identified in the subject. In some embodiments, the sequencing assay used to generate the first set, second set, or third set of sequence read data comprises a whole exome sequencing method. In some embodiments, the sequencing assay used to generate the first set, second set, or third set of sequence read data comprises a whole genome sequencing assay. In some embodiments, each set of sequence read data comprises sequence read data derived from a methylation sequencing method. In some embodiments, the methylation sequencing method comprises use of a bisulfite conversion reaction or an enzymatic conversion reaction to convert non-methylated cytosine to uracil. In some embodiments, the methylation sequencing method comprises use of a chemical or enzymatic conversion reaction to convert methylated cytosine to uracil.

In some embodiments, the methylation sequencing method comprises use of an oxidative bisulfite or enzymatic reaction to detect hydroxymethylation (hmC). In some embodiments, at least one set of sequence read data comprises sequence read data derived from a methylation microarray method. In some embodiments, the disease signal comprises a minimum residual disease (MRD) signal. In some embodiments, the disease signal comprises a tumor response monitoring (TRM) signal. In some embodiments, the disease signal comprises a transplant rejection signal. In some embodiments, the first sample comprises a tumor tissue sample. In some embodiments, the disease-associated DNA comprises circulating tumor DNA (ctDNA).

Disclosed herein are methods for detecting cancer in a subject, the method comprising: a) receiving, at one or more processors, a first set of sequence read data for a plurality of sequence reads derived from a first sample from the subject; b) determining, using the one or more processors, a methylation status for each of a plurality of genomic loci based on the first set of sequence read data; c) identifying, using the one or more processors, a subset of the plurality of genomic loci that are differentially methylated in the first sample based on a comparison of their methylation status to one or more predetermined methylation status thresholds; d) receiving, at one or more processors, methylation status data for the subset of differentially methylated genomic loci in a liquid biopsy sample from the subject; and e) detecting, using the one or more processors, a presence of the cancer in the subset based on the methylation status data for the subset of differentially methylated genomic loci in the liquid biopsy sample.

In some embodiments, the methylation status data for the subset of differentially methylated genomic loci is generated using a PCR-based detection assay, a quantitative PCR-based detection assay, a digital droplet PCR-based detection assay, a methylation-specific PCR-based detection assay, or a microarray-based DNA methylation profiling assay. In some embodiments, the method further comprises estimating a circulating tumor DNA fraction for the first sample and/or the liquid biopsy sample based on the methylation status data for the subset of differentially methylated genomic loci.

In some embodiments, the estimation of circulating tumor DNA fraction is based on a fraction of the subset of differentially methylated genomic loci for which methylation is detected, or an average level of methylation for the subset of differentially methylated genomic loci.

In some embodiments, the method further comprises: receiving, at the one or more processors, a second set of sequence read data for a plurality of sequence reads derived from a control sample from the subject; determining, using the one or more processors, a methylation status for each of the plurality of genomic loci based on the second set of sequence read data; and excluding genomic loci from the subset of differentially methylated genomic loci identified in step 74 (c) based on a comparison of their methylation status determined based on the second set of sequencing data to the one or more predetermined methylation status thresholds.

In some embodiments, the control sample comprises a matched normal tissue sample from the subject. In some embodiments, the control sample comprises a buffy coat sample from the subject. In some embodiments, the detection of the presence of cancer in the subject further comprises detection of one or more somatic single base substitutions, indels, or structural variants based on the first set of sequence read data or an analysis of the liquid biopsy sample.

In some embodiments, the plurality of genomic loci comprises at least 10, at least 100, at least 1,000, at least 5,000, at least 10,000, at least 20,000, at least 30,000, at least 40,000, at least 50,000, at least 60,000, at least 70,000, at least 80,000, at least 90,000, at least 100,000, at least 120,000, at last 140,000, at least 160,000, at least 180,000, or at least 200,000 genomic loci.

In some embodiments, the identified subset of the plurality of genomic loci is tumor-specific for a subject that has been diagnosed with cancer. In some embodiments, the identified subset of the plurality of genomic loci is treatment-specific for a subject that has previously been treated with an anti-cancer therapy. In some embodiments, the identified subset of the plurality of genomic loci is patient-specific.

In some embodiments, the one or more predetermined methylation status thresholds are determined based on an average degree of methylation detected at a specified plurality of genomic loci in a cohort of healthy and diseased subjects. In some embodiments, the one or more predetermined methylation status thresholds are determined based on an analysis of cluster consensus methylation fractions (CCMFs) for a cohort of healthy and diseased subjects. In some embodiments, the one or more predetermined methylation status thresholds are determined based on an analysis of cluster consensus unmethylation fractions (CCUFs) for a cohort of healthy and diseased subjects.

In some embodiments, determining a cluster consensus methylation fraction (CCMF) or a cluster consensus unmethylation fraction (CCUF) for each of the subjects in the cohort of heathy and diseased subjects comprises: receiving, at one or more processors, sequence read data for a plurality of sequence reads derived from a sample from a subject, wherein at least a portion of the plurality of sequence reads corresponds to a genomic locus comprising a cluster of two or more CpG dinucleotides; determining, using the one or more processors, a consensus methylation pattern or a consensus unmethylation pattern for the cluster, wherein the consensus methylation pattern represents each CpG dinucleotide in the cluster for which methylation was detected based on the absence cytosine conversion in at least one sequence read from the sequence read data, and wherein the consensus unmethylation pattern represents each CpG dinucleotide in the cluster for which methylation was not detected; generating, using the one or more processors, a cluster consensus fraction (CCF) for the cluster, wherein the CCF represents a fraction of sequence reads corresponding to the cluster that show the consensus methylation pattern, or a fraction of sequence reads corresponding to the cluster that show the consensus unmethylation pattern, out of a total number of sequence reads from the plurality corresponding to the cluster; and detecting one or more of the methylation level or the unmethylation level of the cluster based on the CCF.

In some embodiments, the one or more predetermined methylation status thresholds are determined for the subject based on a comparison of the methylation status for each of the plurality of genomic loci as determined based on the first set of sequence read data and as determined based on a set of sequence read data derived from a control sample from the subject. In some embodiments, the control sample a matched normal tissue sample from the subject.

In some embodiments, the methylation status of a given genomic locus of the plurality of genomic loci comprises a hypermethylation status, a hypomethylation status, or a hemimethylation status. In some embodiments, the liquid biopsy sample comprises blood, plasma, serum, cerebrospinal fluid, sputum, stool, urine, or saliva.

In some embodiments, the method further comprises performing a sequencing assay to generate the first set of sequence read data. In some embodiments, the sequencing assay used to generate the first set of sequence read data comprises a targeted sequencing method. In some embodiments, the targeted sequencing method comprises use of a set of bait molecules designed to target the subset of differentially methylated genomic loci. In some embodiments, the set of bait molecules is customized to target a set of differentially methylated genomic loci identified in the subject. In some embodiments, the methylation status data for the subset of differentially methylated genomic loci is generated using a PCR-based detection assay, a quantitative PCR-based detection assay, a digital droplet PCR-based detection assay, or a methylation-specific PCR-based detection assay and a custom set of PCR primers designed to target a set of differentially methylated genomic loci identified in the subject.

Disclosed herein are methods for diagnosing a disease, the method comprising: diagnosing that a subject has the disease based on a determination of personalized methylation biomarkers for a sample from the subject, wherein the personalized methylation biomarkers are determined according to the methods described above.

Disclosed herein are methods of selecting an anti-cancer therapy, the method comprising: responsive to determining personalized methylation biomarkers for a sample from a subject, selecting an anti-cancer therapy for the subject, wherein the personalized methylation biomarkers are determined according to the method described above.

Disclosed herein are methods of treating a cancer in a subject, the method comprising: responsive to determining personalized methylation biomarkers for a sample from the subject, administering an effective amount of an anti-cancer therapy to the subject, wherein the personalized methylation biomarkers is determined according to the method of any one of methods above.

Disclosed herein are methods for monitoring cancer progression or recurrence in a subject, the method comprising: detecting a first disease signal corresponding to cancer based on a methylation status determined for a subset of differentially methylated genomic loci identified in sequence read data derived from a sample obtained from the subject at a first time point, where the disease signal is detected according to the methods described above; detecting a second disease signal corresponding to cancer based on a methylation status determined for a subset of differentially methylated genomic loci identified in sequence read data derived from a sample obtained from the subject at a second time point; and comparing the first disease signal to the second disease signal, thereby monitoring the cancer progression or recurrence.

In some embodiments, the second disease signal is detected according to the methods described above. In some embodiments, the method further comprises selecting an anti-cancer therapy for the subject in response to the cancer progression. In some embodiments, the method further comprises administering an anti-cancer therapy to the subject in response to the cancer progression. In some embodiments, the method further comprises adjusting an anti-cancer therapy for the subject in response to the cancer progression. In some embodiments, the method further comprises adjusting a dosage of the anti-cancer therapy or selecting a different anti-cancer therapy in response to the cancer progression. In some embodiments, the method further comprises administering the adjusted anti-cancer therapy to the subject.

In some embodiments, the first time point is before the subject has been administered an anti-cancer therapy, and wherein the second time point is after the subject has been administered the anti-cancer therapy. In some embodiments, the subject has a cancer, is at risk of having a cancer, is being routine tested for cancer, or is suspected of having a cancer. In some embodiments, the cancer is a solid tumor. In some embodiments, the cancer is a hematological cancer. In some embodiments, the anti-cancer therapy comprises chemotherapy, radiation therapy, immunotherapy, a targeted therapy, or surgery.

In some embodiments, the method further comprises determining, identifying, or applying a methylation status value determined for the subset of differentially methylated genomic loci as a diagnostic value associated with the sample. In some embodiments, the method further comprises generating a genomic profile for the subject based on a determination of personalized methylation biomarkers. In some embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.

In some embodiments, the genomic profile for the subject further comprises results from a nucleic acid sequencing-based test. In some embodiments, the method further comprises selecting an anti-cancer therapy, administering an anti-cancer therapy, or applying an anti-cancer therapy to the subject based on the generated genomic profile. In some embodiments, the detection of the disease signal is used in making suggested treatment decisions for the subject. In some embodiments, the detection of the disease signal is used in applying or administering a treatment to the subject.

Disclosed herein are systems comprising one or more processors and a memory communicatively coupled to the one or more processors and configured to store instructions that, when executed by the one or more processors, cause the system to: receive a first set of sequence read data for a plurality of sequence reads derived from a first sample from the subject; determine a methylation status for each of a plurality of genomic loci based on the first set of sequence read data; and identify a subset of the plurality of genomic loci that are differentially methylated in the first sample based on a comparison of their methylation status to one or more predetermined methylation status thresholds.

In some embodiments, the system further comprises instructions that, when executed by the one or more processors, cause the system to: receive a second set of sequence read data for a plurality of sequence reads derived from a liquid biopsy sample from the subject; determine a methylation status for the subset of differentially methylated genomic loci based on the second set of sequence read data; and detect a disease signal in the subject based on the methylation status determined for the subset of differentially methylated genomic loci in the second set of sequence read data, wherein the disease signal is indicative of the disease status of the subject.

Disclosed herein is a non-transitory computer-readable storage medium storing one or more programs, the one or more programs comprising instructions, which when executed by one or more processors of a system, cause the system to: receive a first set of sequence read data for a plurality of sequence reads derived from a first sample from the subject; determine a methylation status for each of a plurality of genomic loci based on the first set of sequence read data; and identify a subset of the plurality of genomic loci that are differentially methylated in the first sample based on a comparison of their methylation status to one or more predetermined methylation status thresholds.

In some embodiments, the non-transitory computer-readable storage medium further comprises instructions that, when executed by the one or more processors of a system, cause the system to: receive a second set of sequence read data for a plurality of sequence reads derived from a liquid biopsy sample from the subject; determine a methylation status for the subset of differentially methylated genomic loci based on the second set of sequence read data; and detect a disease signal in the subject based on the methylation status determined for the subset of differentially methylated genomic loci in the second set of sequence read data, wherein the disease signal is indicative of the disease status of the subject.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 depicts a non-limiting exemplary method for tracking personalized methylation biomarkers for the detection of disease, in accordance with some embodiments of the present disclosure.

FIG. 2 depicts an exemplary computing device or system, in accordance with some embodiments of the present disclosure.

FIG. 3 depicts an exemplary computer system or computer network, in accordance with some instances of the systems described herein.

FIG. 4 depicts a non-limiting example of data that illustrates clinically relevant details about the biological samples from which sequence read data are derived, in accordance with some embodiments of the present disclosure.

FIG. 5 depicts a non-limiting exemplary workflow for the generation of sequence read data, in accordance with some embodiments of the present disclosure.

FIG. 6 depicts a non-limiting exemplary schematic showing a process for generating a personalized methylation signature for an individual, based on an analysis of methylation sequence data derived from a tumor tissue sample and a blood-derived (e.g., Buffy coat) sample, in accordance with some embodiments of the present disclosure.

FIGS. 7A-7C depict a non-limiting example of data for the fraction of biomarkers identified in patient plasma samples for different cancer types that exhibit methylation signals. The different panels indicate the fraction of biomarkers that exhibit methylation signals before and after personalization of the biomarkers based on methylation statuses for DNA extracted from patient-derived formaldehyde-fixed paraffin-embedded (FFPE) tissue samples, in accordance with some embodiments of the present disclosure. FIG. 7A depicts the fraction of biomarkers that exhibit methylation signals before being subjected to personalization methods, as detailed in the present disclosure. FIG. 7B depicts the fraction of biomarkers that exhibit methylation signals after being subjected to personalization methods, as detailed in the present disclosure. FIG. 7C depicts the fraction of biomarkers that exhibit methylation signals after methylation sites that are present in healthy buffy coat data are excluded and the remaining methylation sites are subject to personalization methods, as detailed in the present disclosure.

FIGS. 8A-8B provide non-limiting examples of tumor fraction (TF) data for a variety of lung cancer samples determined using the methods disclosed herein. FIG. 8A: data for early-stage lung cancer patients. FIG. 8B: data for late-stage lung cancer patients.

FIG. 9 provides a non-limiting example of disease detection rates for the methods disclosed herein and their correlation with tumor mutational burden (TMB).

FIG. 10 provides a non-limiting example of data for the distributions of the number of hypermethylated loci (left panel) and hypomethylated loci (right panel) that were observed for three types of cancer.

FIG. 11 provides a non-limiting example of data illustrating that tracking personalize methylation biomarkers provides for informed estimates of tumor fraction.

### DETAILED DESCRIPTION

Disclosed herein are methods and systems for tracking personalized methylation biomarkers for the detection of disease. Personalized methylation biomarkers can be identified in DNA extracted from patient tissue samples, and subsequently tracked in cell-free DNA (cfDNA) extracted from matched patient plasma samples. Existing methods that analyze cfDNA in order to detect ctDNA often suffer from lack of detection sensitivity and/or interference from CHIP, as noted above. By first characterizing a set of personalized methylation biomarkers (e.g., a set of differentially methylated variants) in high signal tumor tissue, the disclosed methods are less prone to specificity errors and improve ctDNA detection limits. The disclosed methods may be less susceptible to sensitivity issues for samples with small numbers having somatic sequence variants since methylation status aberrations are common and may recur in similar locations between patients. The disclosed methods are also less susceptible to CHIP interference, because, in general, methylation of cfDNA derived from CHIP cfDNA may be far less common than methylation of ctDNA in cancer-associated methylation regions. Furthermore, the methods and systems described herein benefit from the advantages generally seen in liquid biopsy-based, noninvasive cfDNA analysis and ctDNA detection schemes.

The methods of the present disclosure aim to use ctDNA methylation statuses, rather than the genomic sequence of the ctDNA, to detect, for example, the presence of even low levels of tumor-derived DNA and thereby better assess a patient's disease risk, e.g., recurrence risk. The disclosed methods enable enhanced detection sensitivity for ctDNA by: (i) focusing on detecting methylation status for a personalized sets of differentially-methylated genomic loci (as identified based on DNA extracted from patient tissue samples) that minimize baseline signal, (ii) by evaluating large numbers of differentially-methylated genomic loci identified for a given patient (thereby increasing methylation signals), and (iii) optionally eliminating from consideration genomic loci associated with variants that likely arose through CHIP.

In some instances, for example, the disclosed methods for detection of a disease signal in a subject may comprise: a) receiving a first set of sequence read data for a plurality of sequence reads derived from a first sample (e.g., a tissue sample) from the subject; b) determining a methylation status for each of a plurality of genomic loci based on the first set of sequence read data; c) identifying a subset of the plurality of genomic loci that are differentially methylated in the first sample based on a comparison of their methylation status to one or more predetermined methylation status thresholds; d) receiving a second set of sequence read data for a plurality of sequence reads derived from a second sample (e.g., a liquid biopsy sample) from the subject; e) determining a methylation status for the subset of differentially methylated genomic loci based on the second set of sequence read data; and f) detecting the disease signal in the subject based on the methylation status determined for the subset of differentially methylated genomic loci in the second set of sequence read data.

In some instances, the method may further comprise estimating a disease-associated DNA fraction for the second sample (e.g., a liquid biopsy sample) based on the methylation status determined at the subset of differentially methylated genomic loci in the second set of sequence read data.

### DEFINITIONS

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type”sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Methods for Tracking Personalized Methylation Biomarkers for the Detection of Disease

Disclosed herein are methods and systems for tracking personalized methylation biomarkers for the detection of disease. Personalized methylation biomarkers can be identified in DNA extracted from patient tissue samples, and subsequently tracked in cell-free DNA (cfDNA) extracted from matched patient plasma samples. Existing methods that analyze cfDNA in order to detect ctDNA often suffer from lack of detection sensitivity and/or interference from CHIP, as noted above. By first characterizing a set of personalized methylation biomarkers (e.g., a set of differentially methylated variants) in high signal tumor tissue, the disclosed methods are less prone to specificity errors and improve ctDNA detection limits. The disclosed methods are also less susceptible to CHIP interference, because, in general, methylation of CHIP variants is far less common than methylation of ctDNA. Furthermore, the methods and systems described herein benefit from the advantages generally seen in liquid biopsy-based, noninvasive cfDNA analysis and ctDNA detection schemes.

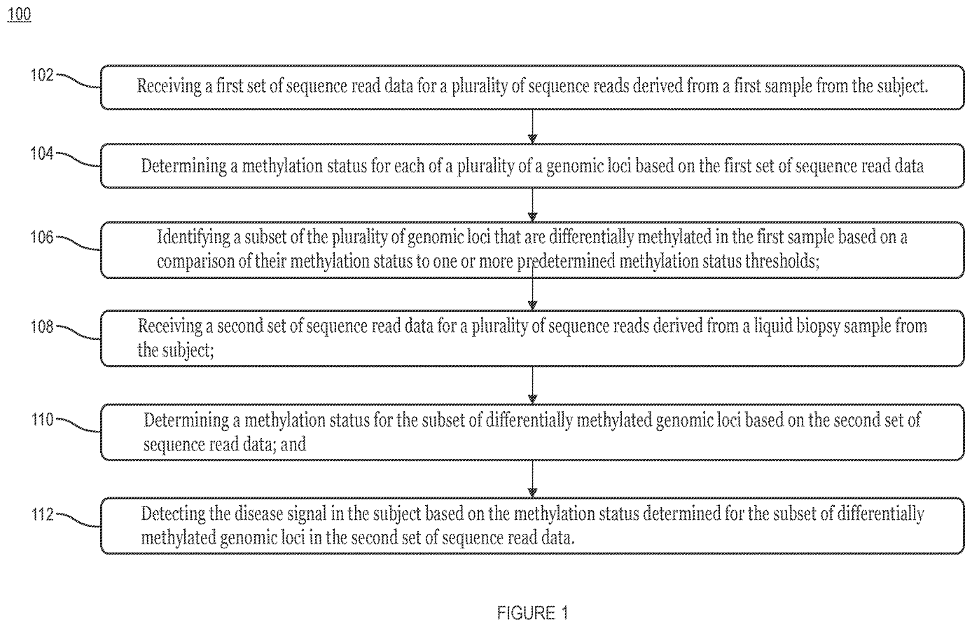

FIG. 1. illustrates an exemplary schematic showing a general process 100 for generating a set of personalized methylation biomarkers for a subject and using them for the detection of disease (e.g., cancer). Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, a first set of sequence read data for a plurality of sequence reads derived from a first sample from the subject is received.

In some instances, the first sample from the subject may comprise, e.g., a tissue sample, a tissue biopsy sample, or a tumor sample.

In some instances, a sequencing assay can be used to generate the first set of sequence read data, and/or other sets of sequence read data, such as a second set of sequence read data or a third set of sequence read data. In some instances, the sequencing assay used to generate sequence read data, such as a first, second, or third set of sequence read data, can comprise, for example, a methylation microarray assay, a whole genome methylation sequencing assay, or a targeted methylation sequencing method, a methylation selection method such as immunoprecipitation of methylated sequence followed by sequencing, or methylation-specific PCR or methylation-specific ddPCR. The targeted sequencing method used to generate a set of sequence read data can comprise the use of bait molecules designed to target a plurality of genomic loci. In some instances, the targeted sequencing method can comprise the use of bait molecules designed to target a subset of the plurality of genomic loci, e.g., the subset of differentially methylated genomic loci identified in step 106 in FIG. 1 as described below. In some instances, the bait molecules can be customized to target a specified set of differentially methylated genomic loci identified in the subject.

In some instances, the first set of sequence read data, as well as other sets of sequence read data, can derive from a methylation sequencing method that comprises the use of, e.g., a bisulfite conversion reaction which converts non-methylated cytosine to uracil, an enzymatic conversion reaction which converts non-methylated cytosine to uracil, a chemical or enzymatic conversion reaction which converts methylated cytosine to uracil, or an oxidative bisulfite or enzymatic reaction which detects hydroxymethylation (hmC).

At step 104 in FIG. 1, a methylation status is determined for each of a plurality of a genomic loci based on the first set of sequence read data.

In some instances, the methylation status of a given genomic locus of the plurality of genomic loci can comprise a hypermethylation status, a hypomethylation status, or a hemimethylation status. A genomic locus may contain a single CpG or multiple CpGs that are all differentially methylated in concert. In some examples, a genomic locus might contain Cs that have methylation present at Cs not present in a CpG context, for example, a CA sequence of nucleotides.

In some instances, the plurality of genomic loci are identified based on an analysis of methylation status at genomic loci in one or more samples from healthy individuals.

In some instances, the plurality of genomic loci can comprise at least 10, at least 100, at least 1,000, at least 5,000, at least 10,000, at least 20,000, at least 30,000, at least 40,000, at least 50,000, at least 60,000, at least 70,000, at least 80,000, at least 90,000, at least 100,000, at least 120,000, at least 140,000, at least 160,000, at least 180,000, at least 200,000, at least 400,000, at least 600,000, at least 800,000, at least 1,000,000, at least 1,500,000, at least 2,000,000, at least 2,500,000, or at least 3,000,000 genomic loci. In some instances, the plurality of genomic loci can comprise any number of genomic loci within this range of values.

At step 106 in FIG. 1, a subset of the plurality of genomic loci that are differentially methylated in the first sample is identified based on a comparison of their methylation status to one or more predetermined methylation status thresholds.

In some instances, the subset of the plurality of genomic loci that are differentially methylated can comprise at least 10, at least 100, at least 1,000, at least 5,000, at least 10,000, at least 20,000, at least 30,000, at least 40,000, at least 50,000, at least 60,000, at least 70,000, at least 80,000, at least 90,000, at least 100,000, at least 120,000, at least 140,000, at least 160,000, at least 180,000, or at least 200,000, at least 400,000, at least 600,000, at least 800,000, at least 1,000,000, at least 1,500,000, at least 2,000,000, at least 2,500,000, or at least 3,000,000 genomic loci. In some instances, the subset of the plurality of genomic loci that are differentially methylated can comprise any number of genomic loci within this range of values.

In some instances, the identified subset of the plurality of genomic loci can be disease-specific for a subject that has been diagnosed with a disease. In some instances, the identified subset of the plurality of genomic loci can be tumor-specific for a subject that has been diagnosed with cancer, or a particular kind of cancer, treatment-specific for a subject that has previously been treated with an anti-cancer therapy, or is patient-specific.

In some instances, the one or more predetermined methylation status thresholds can be determined based on a) an average degree of methylation detected at a specified plurality of genomic loci in a cohort of healthy and diseased subjects; b) an analysis of cluster consensus methylation fractions (CCMFs) for a cohort of healthy and diseased subjects; or c) an analysis of cluster consensus unmethylation fractions (CCUFs) for a cohort of healthy and diseased subjects.

In some instances, the determination of at least one CCMF or at least one CCUF for each of the subjects in the cohort of healthy and diseased subjects can comprise a) receiving sequence read data for a plurality of sequence reads derived from a sample from a subject, where at least a portion of the plurality of sequence reads corresponds to a genomic locus comprising a cluster of two or more CpG dinucleotides; b) determining a consensus methylation pattern or a consensus unmethylation pattern for the cluster, where the consensus methylation pattern represents each CpG dinucleotide in the cluster for which methylation was detected based on the absence cytosine conversion in at least one sequence read from the sequence read data, and where the consensus unmethylation pattern represents each CpG dinucleotide in the cluster for which methylation was not detected; c) generating a cluster consensus fraction (CCF) for the cluster, wherein the CCF represents a fraction of sequence reads corresponding to the cluster that shows the consensus methylation pattern, or a fraction of sequence reads corresponding to the cluster that shows the consensus unmethylation pattern, out of a total number of sequence reads from the plurality corresponding to the cluster; and d) detecting one or more of the methylation levels or the unmethylation levels of the cluster, based on the CCF.

In some instances, the one or more predetermined methylation status thresholds determined for the subject can be based on a comparison of the methylation status for each of the plurality of genomic loci as determined based on the first set of sequence read data and as determined based on a set of sequence read data derived from a control sample from the subject. In some instances, the control sample can be, for example, a matched normal tissue sample from the subject.

At step 108 in FIG. 1, a second set of sequence read data for a plurality of a sequence reads derived from a second sample, e.g., a liquid biopsy sample, from the subject is received. The liquid biopsy sample can comprise, for example, blood, plasma, serum, cerebrospinal fluid, sputum, stool, urine, or saliva.

At step 110 in FIG. 1, a methylation status is determined for the subset of differentially methylated genomic loci based on the second set of sequence read data.

In some instances, the methylation status for the subset of differentially methylated genomic loci in the second set of sequence read data can comprise a hypermethylation status, a hypomethylation status, or a hemimethylation status.

In some instances, the methylation status data for the subset of differentially methylated genomic loci is generated using a PCR-based detection assay, a quantitative PCR-based detection assay, a digital droplet PCR-based detection assay, a methylation-specific PCR-based detection assay, a methylation-specific selection assay, such as immunoprecipitation of methylated DNA, or a microarray-based DNA methylation profiling assay.

In some instances, the methylation status for the subset of differentially methylated genomic loci can be used to estimate a circulating tumor DNA fraction for the first sample and/or the liquid biopsy sample (e.g. a second sample). The estimation of the circulating tumor DNA fraction can be based on, for example, a fraction of the subset of differentially methylated genomic loci for which methylation is detected, or an average level of methylation for the subset of differentially methylated genomic loci.

At step 112 in FIG. 1, a disease signal in the subject is detected based on the methylation status determined for the subset of differentially methylated genomic loci in the second set of sequence read data.

In some instances, the detection of the disease signal (e.g., a cancer signal) can also be based on the detection of one or more somatic single base substitutions, indels, or structural variants, in the first set of sequence read data, or an analysis of the liquid biopsy sample (or second sample).

In some instances, the method may further comprise estimating a disease-associated DNA fraction for the liquid biopsy sample (e.g. a second sample) based on the methylation status determined at the subset of differentially methylated genomic loci in the second set of sequence read data. In some instances, the estimation of disease-associated DNA fraction can be based on a fraction of the subset of differentially methylated genomic loci for which methylation is detected, an average level of methylation for the subset of differentially methylated genomic loci, a fraction of differentially methylated sequence reads in the second set of sequence read data, or an absolute number of differentially methylated reads in the second set of sequence read data. In some instances, the disease-associated DNA may comprise circulating tumor DNA (ctDNA).

In some instances, the method may further comprise: a) receiving, at the one or more processors, a third set of sequence read data for a plurality of sequence reads derived from a disease-free control sample from the subject; b) determining, using the one or more processors, a methylation status for each of the plurality of genomic loci based on the third set of sequence read data; and c) excluding genomic loci from the subset of differentially methylated genomic loci identified in step 106, if their methylation status is the same for the first sample and the disease-free control sample. In some instances, the disease-free control sample comprises a matched normal tissue sample from the subject. In some instances, the disease-free control sample comprises a buffy coat sample from the subject. In some instances, the disease-free control sample comprises an adjacent normal tissue sample from the subject.

In some instances, for example, the disclosed methods may comprise a method for detection of cancer in a subject comprising: a) receiving, at one or more processors, a first set of sequence read data for a plurality of sequence reads derived from a first sample from the subject; b) determining, using the one or more processors, a methylation status for each of a plurality of genomic loci based on the first set of sequence read data; c) identifying, using the one or more processors, a subset of the plurality of genomic loci that are differentially methylated in the first sample based on a comparison of their methylation status to one or more predetermined methylation status thresholds; d) receiving, at one or more processors, methylation status data for the subset of differentially methylated genomic loci in a liquid biopsy sample (e.g. a second sample) from the subject; and e) detecting, using the one or more processors, a presence of the cancer in the subset based on the methylation status data for the subset of differentially methylated genomic loci in the liquid biopsy sample (e.g. a second sample).

In some instances, the disclosed methods may be used for the detection of minimum residual disease (MRD) or a disease signal associated with MRD.

In some instances, the disclosed methods may be used for the detection of treatment response monitoring (TRM), or a disease signal associated with TRM.

In some instances, the disclosed methods may be used for the detection of transplant rejection, or a disease signal associated with transplant rejection.

The methods of the present disclosure can comprise the use of different sequencing strategies when sequencing the DNA extracted from tumor (e.g., tissue samples or tissue biopsy samples) and/or non-tumor-derived samples (e.g., liquid biopsy samples) from an individual. For example, in some instances a whole genome sequencing method can be used so that the methylation status can be determined at a large number of genomic loci, e.g., up to at least 1,000, 5,000, 10,000, 20,000, 30,000, 40,000, 50,000, 60,000, 70,000, 80,000, 90,000, 100,000, 125,000, 150,000, 175,00, or 200,000 genomic loci, albeit at a low coverage (such as at 30× read depth) in some instances. Alternatively, for example, a hybrid capture method can be used, so that methylation status can be determined at specific target sites in the genome, such as 5,000, 10,000, 20,000, 30,000, 40,000, or 50000 sites can be read at a higher coverage, such as at 4,000× read depth. In some instances, the specific target sites in the genome for which methylation status is evaluated may be established in the art as sites that are known to be hypermethylated in cancer.

Alternatively, in some instances, the methods of the present disclosure can comprise using other sequencing methods to determine the methylation profile of cfDNA from a sample, such as, but not limited to, a methyl-PCR assay or a microarray.

In some instances, the methods of the present disclosure can comprise the use of formalin-fixed paraffin-embedded (FFPE) tumor tissue as the biological sample. DNA can be isolated from FFPE tissue samples using methods including, but not limited to, the Omega BioTek (Norcross, GA) Mag-Bind Particles method.

In some instances, the methods of the present disclosure can comprise the use of a Buffy coat sample as the biological sample. DNA can be isolated from Buffy coat samples using methods including, but not limited to, the Applied Biosystems (Waltham, MA) MagMAX DNA Multi-Sample Ultra 2.0 method.

In some instances, the methods of the present disclosure can comprise the use of plasma, including normal healthy plasma, as the biological sample. DNA can be isolated from plasma samples using methods including, but not limited to, the Apostle (San Jose, CA) MiniMAX High Efficiency cfDNA Isolation method.

In some instances, the method of shearing DNA can comprise the use of methods including, but not limited to, the Covaris (Woburn, MA) nucleic acid shearing method.

In some instances, the library construction protocol can comprise the use of methods including, but not limited to, the NEB (Ipswich, MA) NEBNext Enzymatic Methyl-seq method.

In some instances, the hybrid capture protocol can comprise the use of a kit, including, but not limited to, the Twist Bioscience (South San Francisco, CA) Targeted Methylation Sequencing kit.

In some instances, the sequencing platform can comprise the use of, e.g., an Illumina (San Diego, CA) sequencer and NovaSeq S4 Flow Cells.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome methylation sequencing (WGS) technique, a targeted methylation sequencing technique, a direct sequencing technique, or a Sanger sequencing technique using, e.g., a next-generation (massively parallel) sequencer, and (viii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for tracking personalized methylation biomarkers for the detection of disease may be used to diagnose (or as part of a diagnosis of) the presence of disease or other condition (e.g., cancer, genetic disorders (such as Down Syndrome and Fragile X), neurological disorders, transplant rejection, or any other disease type where detection of variants, e.g., methylation alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for tracking personalized methylation biomarkers for the detection of disease may be used to select a subject (e.g., a patient) for a clinical trial based on, e.g., the methylation status determined for one or more differentially methylated genomic loci. In some instances, patient selection for clinical trials based on, e.g., detection of methylation status at one or more differentially methylated genomic loci, may accelerate the development of targeted therapies and improve the healthcare outcomes for treatment decisions.

In some instances, the disclosed methods for tracking personalized methylation biomarkers for the detection of disease may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for tracking personalized methylation biomarkers for the detection of disease may be used in treating a disease (e.g., a cancer) in a subject. For example, in response to detecting disease (e.g., cancer) based on a set of personalized methylation biomarkers using any of the methods disclosed herein, an effective amount of, e.g., an anti-cancer therapy or anti-cancer treatment may be administered to the subject.

In some instances, the disclosed methods for tracking personalized methylation biomarkers for the detection of disease may be used for monitoring disease progression or recurrence (e.g., cancer or tumor progression or recurrence) in a subject. For example, in some instances, the methods may be used to determine personalized methylation biomarkers for the detection of disease in a first sample obtained from the subject at a first time point, and used to determine personalized methylation biomarkers for the detection of disease in a second sample obtained from the subject at a second time point, where comparison of the first determination of personalized methylation biomarkers for the detection of disease and the second determination of personalized methylation biomarkers for the detection of disease allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of tracking personalized methylation biomarkers for the detection of disease.

In some instances, the set of personalized methylation biomarkers determined using the disclosed methods may be used as a prognostic or diagnostic indicator associated with a patient sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for tracking personalized methylation biomarkers for the detection of disease may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for tracking personalized methylation biomarkers for the detection of disease as part of a genomic profiling process (or inclusion of the output from the disclosed methods for tracking personalized methylation biomarkers for the detection of disease as part of the genomic profile of the subject) can improve the validity of, e.g., disease detection calls and treatment decisions, made on the basis of the genomic profile by, for example, independently confirming the presence of residual disease (e.g., cancer) in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway or homologous recombination deficiency (HRD).

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence or aberrant methylation status as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164 (1): 35-42; Masuda, et al., (1999) Nucleic Acids Res. 27 (22): 4436-4443; Specht, et al., (2001) Am J Pathol. 158 (2): 419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, CpG islands, CpG shores, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the target capture reagent may be designed to select DNA that is methylated or is not methylated. In some instances, a bisulfite conversion reaction or an enzymatic conversion reaction can convert non-methylated cytosine to uracil. In some instances, a chemical or enzymatic conversion reaction can convert methylated cytosine to uracil.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4 (11): 903-5; Hodges, E. et al. (2007) Nat. Genet. 39 (12): 1522-7; and Okou, D. T. et al. (2007) Nat. Methods 4 (11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 103, 104, 105 or more than 105 molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Ultima Genomics sequencing platform, Element Biosciences sequencing platform, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Alignment of Methyl-Seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a bisulfite- or enzymatic-conversion reaction (e.g., during library preparation) to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions (e.g., during library preparation) to convert non-methylated cytosine to uracil (or to convert methylated cytosine to dihydrouracil). For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR amplification converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27 (11): 1571-1572).

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6): 847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ~1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9): 1297-303; Ye, K., et al., Bioinformatics, 2009; 25(21): 2865-71; Lunter, G., and Goodson, M., Genome Res. 2011; 21(6): 936-9; and Li, H., et al. (2009), Bioinformatics 25(16): 2078-9.

Methods for generating indel calls and individual-level genotype likelihoods include, e.g., the Dindel algorithm (Albers, C. A., et al., Genome Res. 2011; 21(6): 961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6): 952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix—Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27(11): 1571-1572), TARGOMICS (Garinet, et al. (2017), “Calling Chromosome Alterations, DNA Methylation Statuses, and Mutations in Tumors by Simple Targeted Next-Generation Sequencing-A Solution for Transferring Integrated Pangenomic Studies into Routine Practice?”, J. Molecular Diagnostics 19(5): 776-787), Bicycle (Grana, et al. (2018) “Bicycle: A Bioinformatics Pipeline to Analyze Bisulfite Sequencing Data”, Bioinformatics 34(8): 1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski J K, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Systems

Also disclosed herein are systems designed to implement any of the disclosed methods for tracking personalized methylation biomarkers for the detection of disease in a sample from a subject. The systems may comprise, e.g., one or more processors, and a memory unit communicatively coupled to the one or more processors and configured to store instructions that, when executed by the one or more processors, cause the system to: receive a first set of sequence read data for a plurality of sequence reads derived from a first sample (e.g., a tissue sample) from the subject; determine a methylation status for each of a plurality of genomic loci based on the first set of sequence read data; identify a subset of the plurality of genomic loci that are differentially methylated in the first sample based on a comparison of their methylation status to one or more predetermined methylation status thresholds; receive a second set of sequence read data for a plurality of sequence reads derived from a second sample (e.g., a liquid biopsy sample) from the subject; determine a methylation status for the subset of differentially methylated genomic loci based on the second set of sequence read data; and detect a disease signal in the subject based on the methylation status determined for the subset of differentially methylated genomic loci in the second set of sequence read data.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for tracking personalized methylation biomarkers for the detection of disease in any of a variety of samples as described herein (e.g., a tissue sample, biopsy sample, hematological sample, or liquid biopsy sample derived from the subject).

In some instances, the plurality of gene loci for which sequencing data is processed to determine tracking personalized methylation biomarkers for the detection of disease may comprise at least 1, 2, 3, 4, 5, 6, 7, 8, 9, 10, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of tracking personalized methylation biomarkers for the detection of disease is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

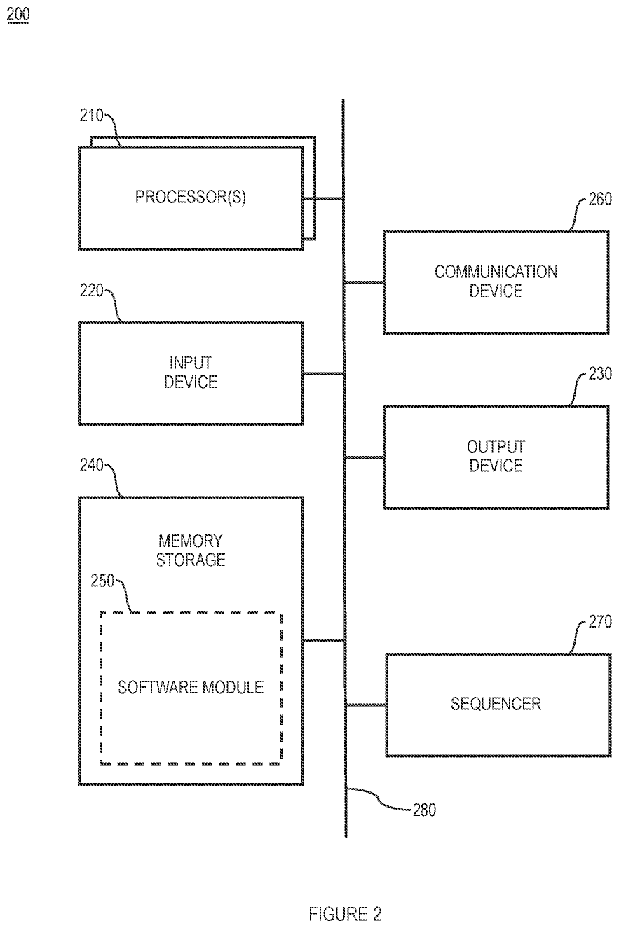

FIG. 2 illustrates an example of a computing device or system in accordance with one embodiment. Device 200 can be a host computer connected to a network. Device 200 can be a client computer or a server. As shown in FIG. 2, device 200 can be any suitable type of microprocessor-based device, such as a personal computer, workstation, server or handheld computing device (portable electronic device) such as a phone or tablet. The device can include, for example, one or more processor(s) 210, input devices 220, output devices 230, memory or storage devices 240, communication devices 260, and nucleic acid sequencers 270. Software 250 residing in memory or storage device 240 may comprise, e.g., an operating system as well as software for executing the methods described herein. Input device 220 and output device 230 can generally correspond to those described herein, and can either be connectable or integrated with the computer.

Input device 220 can be any suitable device that provides input, such as a touch screen, keyboard or keypad, mouse, or voice-recognition device. Output device 230 can be any suitable device that provides output, such as a touch screen, haptics device, or speaker.

Storage 240 can be any suitable device that provides storage (e.g., an electrical, magnetic or optical memory including a RAM (volatile and non-volatile), cache, hard drive, or removable storage disk). Communication device 260 can include any suitable device capable of transmitting and receiving signals over a network, such as a network interface chip or device. The components of the computer can be connected in any suitable manner, such as via a wired media (e.g., a physical system bus 280, Ethernet connection, or any other wire transfer technology) or wirelessly (e.g., Bluetooth®, Wi-Fi®, or any other wireless technology).

Software module 250, which can be stored as executable instructions in storage 240 and executed by processor(s) 210, can include, for example, an operating system and/or the processes that embody the functionality of the methods of the present disclosure (e.g., as embodied in the devices as described herein).

Software module 250 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 240, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

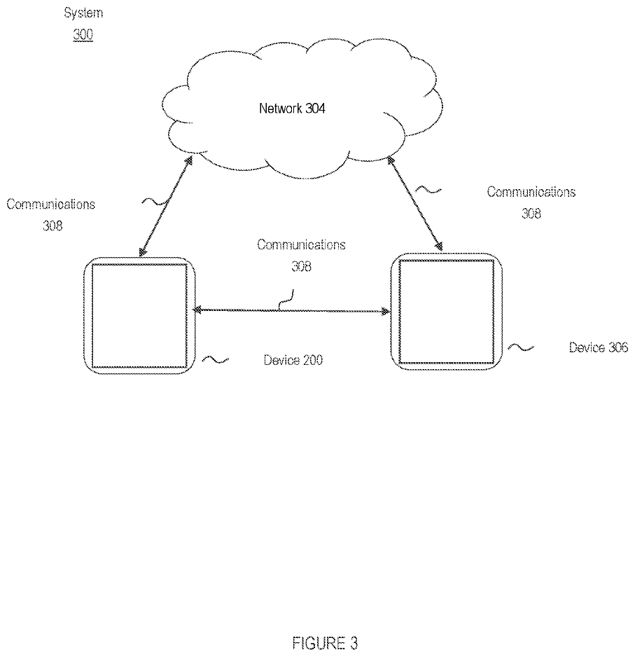

Device 200 may be connected to a network (e.g., network 304, as shown in FIG. 3 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 200 can be implemented using any operating system, e.g., an operating system suitable for operating on the network. Software module 250 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 210.

Device 200 can further include a sequencer 270, which can be any suitable nucleic acid sequencing instrument.

FIG. 3 illustrates an example of a computing system in accordance with one embodiment. In system 300, device 200 (e.g., as described above and illustrated in FIG. 2) is connected to network 304, which is also connected to device 306. In some embodiments, device 306 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 200 and 306 may communicate, e.g., using suitable communication interfaces via network 304, such as a Local Area Network (LAN), Virtual Private Network (VPN), or the Internet. In some embodiments, network 304 can be, for example, the Internet, an intranet, a virtual private network, a cloud network, a wired network, or a wireless network. Devices 200 and 306 may communicate, in part or in whole, via wireless or hardwired communications, such as Ethernet, IEEE 802.11b wireless, or the like. Additionally, devices 200 and 306 may communicate, e.g., using suitable communication interfaces, via a second network, such as a mobile/cellular network. Communication between devices 200 and 306 may further include or communicate with various servers such as a mail server, mobile server, media server, telephone server, and the like. In some embodiments, Devices 200 and 306 can communicate directly (instead of, or in addition to, communicating via network 304), e.g., via wireless or hardwired communications, such as Ethernet, IEEE 802.11b wireless, or the like. In some embodiments, devices 200 and 306 communicate via communications 308, which can be a direct connection or can occur via a network (e.g., network 304).

One or all of devices 200 and 306 generally include logic (e.g., http web server logic) or are programmed to format data, accessed from local or remote databases or other sources of data and content, for providing and/or receiving information via network 304 according to various examples described herein.

### EXAMPLES

### Example 1—A Non-Limiting Example of Data that Illustrates Clinically Relevant Details about the Biological Samples from which Sequence Read Data are Derived, and the Analyses of Those Data

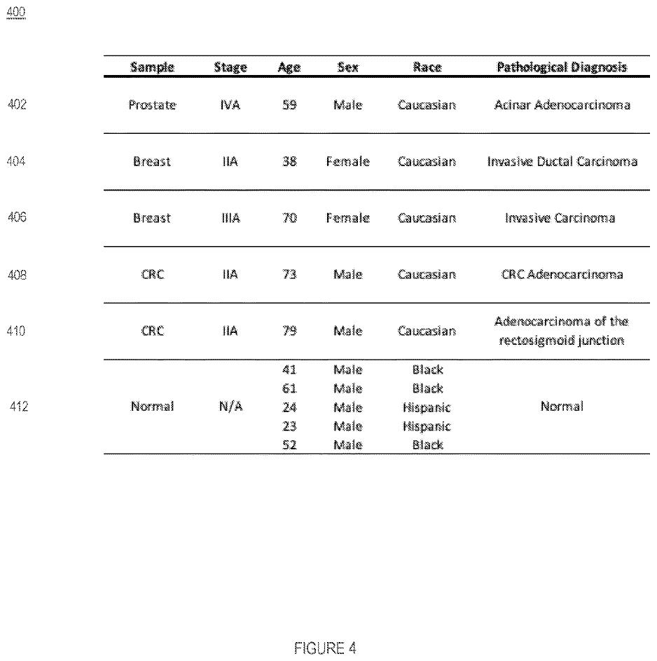

FIG. 4 illustrates a non-limiting example of clinically relevant data about the biological samples from which sequence read data are derived, in the format of table 400, which consists of columns, from left to right, for sample type, cancer stage, age of patient, sex of patient, race of patient, and the pathological diagnosis. Row 402 of table 400 details a prostate sample from a 59 year old Caucasian male diagnosed with stage IVA acinar adenocarcinoma. Row 404 of table 400 details a breast sample from a 38 year old Caucasian female diagnosed with stage IIA invasive ductal carcinoma. Row 406 of table 400 details breast sample from a 70 year old Caucasian female diagnosed with stage IIIA invasive carcinoma. Row 408 of table 400 details a colorectal cancer sample from a 73 year old Caucasian male diagnosed with stage IIA colorectal cancer adenocarcinoma. Row 410 of table 400 details a colorectal cancer sample from a 79 year old Caucasian male diagnosed with stage IIA adenocarcinoma of the rectosigmoid junction. Row 412 of table 400 details a group of normal samples from a 41 year old black male, a 61 year old black male, a 24 year old Hispanic male, a 23 year old Hispanic male, and a 52 year old black male, without any cancer-related pathological diagnoses.

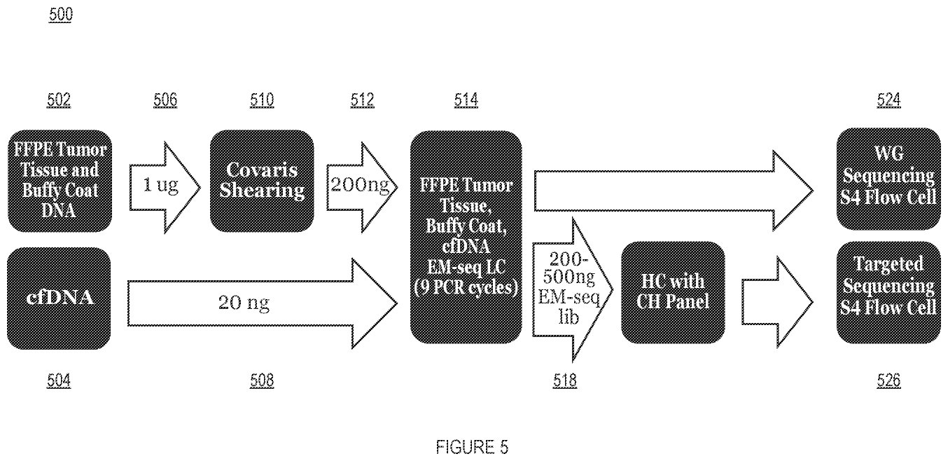

FIG. 5 illustrates a non-limiting exemplary workflow 500 for the generation of sequence read data, for samples such as those described in FIG. 4. In the case of FFPE tumor tissue and buffy coat DNA samples 502, 1 μg of DNA sample 506 is provided for Covaris shearing 510. Then, 200 ng of sheared DNA 512 is subject to EM-seq LC (9 PCR cycles) 514, from which whole genome sequencing is performed on an S4 flow cell 524. In the case of cfDNA samples 504, 20 ng of cfDNA sample 508 is subject to EM-seq LC (9 PCR cycles) 514, from which hybrid capture (HC) with cancer methylation (CM) panel 520 is performed on a targeted sequencing S4 flow cell 526.

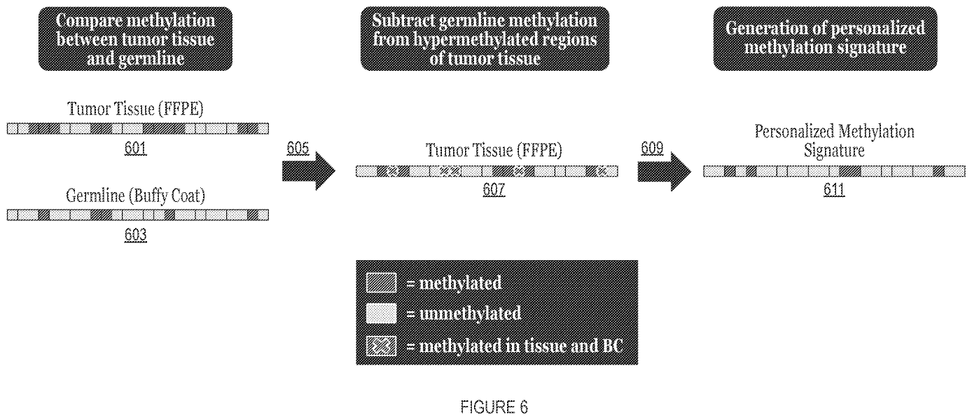

FIG. 6 illustrates a non-limiting exemplary schematic showing a process for deriving a patient's personalized ctDNA methylation status, from samples such as those described in FIG. 4. The methods of the present disclosure aim to compare cell-free DNA (cfDNA) methylation statuses of both tumor sample-derived DNA 601, such as ctDNA from a formalin-fixed and paraformaldehyde embedded (FFPE) tumor sample, and non-tumor-derived cfDNA, where a non-limiting example of a non-tumor-derived cfDNA sample is from the buffy coat 603 that derives from the same individual from which the tumor sample is also derived. The methylation status from the buffy coat cfDNA 603 can represent an individual's non-tumour-derived cfDNA methylation status, such as, but not limited to, methylation statuses that are derived from CHIP, or other processes that are not necessarily carcinogenic. An example analysis from the methods of the present disclosure is to apply a subtraction operation 605, such that an individual's tumor sample methylation status 601 is subtracted by the same individual's buffy coat methylation status 603, to yield a differentiated methylation signature 607, which can be computationally simplified via operation 609 to the differentiated methylation signature 611, which can be considered to be personalized to the individual. An example analysis, like the subtraction operation 605 can yield methylation sites that most likely arise predominantly from the progression of the individual's specific tumor, rather than methylation sites that exist naturally in the individual, from non-disease-related processes. In other words, the methods of the present disclosure can provide a way of identifying personalized methylation biomarkers from an individual's biological sample.

### Example 2—A Non-Limiting Example of Data that Illustrates the Effects of Different Normalization Techniques on the Fraction of Biomarkers from Patient-Derived FFPE Tissues from Different Cancers

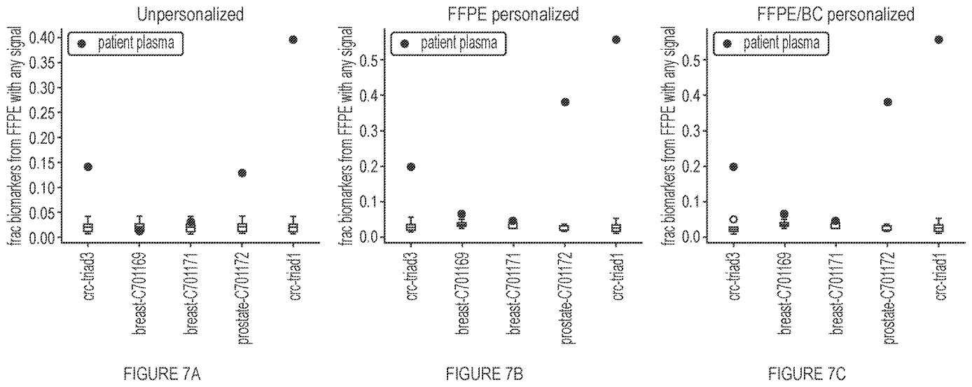

FIGS. 7A-C depict a non-limiting example of data for the fraction of biomarkers identified in patient plasma samples for different cancer types that exhibit methylation signals. The different panels indicate the fraction of biomarkers that exhibit methylation signals before and after personalization of the biomarkers based on methylation statuses for DNA extracted from patient-derived formaldehyde-fixed paraffin-embedded (FFPE) tissue samples. The x-axes for the plots in FIGS. 7A-C are categorical and represent plasma samples (e.g., plasma samples from cancer patients and healthy controls), and the y-axes for the plots in FIGS. 7A-C indicate the fraction of biomarkers identified in the plasma samples that possess a methylation signal. FIG. 7A shows that some plasma ctDNA signal is already visible in three of the five samples, even without any kind of normalization techniques. FIG. 7B shows that when the methods of the present disclosure are applied, such that only patient-specific methylation sites are examined, two samples that were previously in the healthy range, are now detectable, because they are now outside the healthy range, and that the samples that were already detectable, remain detectable, but with improved signal to noise ratios. FIG. 7C shows modest improvements when the methylation sites that are seen in Buffy coat data are excluded from the analysis of the FFPE-derived samples. In FIG. 7C, patient-matched healthy plasma, e.g. after a successful resection, is not used, but if such data were to be used, improved signal to noise ratios can be expected. The depicted FIG. 7C uses non-patient-matched healthy plasma as a stand-in for the preferred patient-matched healthy plasma.

### Example 3—A Non-Limiting Example of Next Generation MRD Detection Assays

The goal for this study was to compare the performance of tissue naïve and tissue informed approaches to using next generation sequencing-based methylation data to detect MRD in liquid biopsy assays. The tissue naïve approach comprised a single-step assay workflow that utilized both genomic and methylation biomarkers identified in targeted sequencing data to detect MRD. The tissue informed approach comprised a two-step, single nucleotide variant (SNV) tracking assay workflow (as described elsewhere herein) that utilized genomic biomarkers identified in whole genome sequencing data to detect MRD.

Next generation MRD (ngMRD) assays need to: (i) be more sensitive than current commercially-available assays, (ii) provide tissue informed and tissue naïve options, and (iii) be supported by clinical evidence in lung cancer, colorectal cancer (CRC), high risk breast cancer, and bladder cancer.

Potential advantages associated with the tissue naïve and tissue-informed assays are outlined in Table 1. In the tumor naïve approach, the use of both genomic and methylation biomarker signals enabled improved detection of sample heterogeneity and increased sensitivity pan-cancer. The reliance on genomic variants identified in whole genome sequencing data in the tissue informed assay enabled improved specificity and maximum sensitivity in high TMB samples.

TABLE 1
Comparison of tissue naïve and tissue informed NGS MRD assays.

| Tissue Naïve | Tissue Informed |
|---|---|
| Hybrid capture increases sensitivity | Maximum sensitivity in high TMB |
| | setting |
| Detect tumor mutational burden | Logistically simple compared to |
| (TMB) high and low cases | bespoke approaches |
| Better logistics relative to the | Drive additional research leveraging |
| tissue informed approach | whole genome information |
| | (e.g., mutational signatures, |
| | complete genomic profiling, |
| | polygenic risk scores (PRS), etc.) |
| Sensitivity gained from: | Leverage existing product knowledge |
| sequencing depth and multiple | |
| signals (genomics biomarkers, | |
| hypermethylation biomarkers, and | |
| hypomethylation biomarkers) | |
| | Sensitivity gained from: sequencing |
| | breadth and very low background |

A cohort of 96 participants (enriched for lung cancer patients) was selected for assay evaluation. The affected patients (n=48) included 29 lung cancer patients, 10 colon/rectal cancer patients, and 9 breast cancer patients, where the lunger cancer patients included:
15 stage I/II patients, 12 stage III patients, and 2 stage IV patients
18 adenocarcinoma patients, 10 squamous patients, and 1 neuroendocrine patient
Smoking status was known for 22 of the 29 patient samples (75%)
35% of the lung cancer patients were former and current smokers

The unaffected patients (n=48) included:
A majority female population (33 of 48 patients (69%))
Smoking status was known in 45 of the 48 cases (69% have never smoked)
Smoking history is enriched in the male population.

With respect to disease ontology (DO) selection, lung cancer was chosen as the primary DO of interest since the assay already baits for biomarkers associated with lung cancer. The target capture panel (bait set) used for the single-step assay workflow is directed to a number of lung cancer biomarkers and methylation biomarkers that overlap with breast and colon cancer biomarkers (e.g., HER2).

To ensure data quality, manual processing was performed for 480 libraries (240 two-step assay workflow libraries, and 240 single-step assay workflow libraries). Three triads (plasma DNA, buffy coat, and solid tumor samples) were eliminated from both approaches due to SNP-identified sample swaps (i.e., vendor swaps).

Tissue sample failure rates were comparable for the two assays, e.g., 5 of the 48 tissue samples for the two-step assay workflow failed due to low sequencing coverage. Similarly, 5 of the 48 tissue samples for the single-step assay workflow also failed.

Whole genome data for buffy coat samples had high coverage variability in the two-step assay workflow (possibly due to a sonication issue), however the data for 40 triads was still useable. For the single-step assay workflow, 44 triads passed data quality control (all affected). Forty seven plasma/buffy coat pairs were processed for each of the affected patients, and forty eight plasma/buffy coat pairs were processed for each of the unaffected patients.

The strategy for evaluating tumor fraction (TF) and background in the two-step assay approach comprised defining a baseline for background tumor fraction using data for formalin-fixed, paraffin-embedded (FFPE) and buffy coat samples, and then looking for DNA fragments in plasma samples that supported the determined baseline for background subtraction (baseline subs).

Tumor fraction was evaluated using two different approaches. In the first approach, tumor fraction (TF₁)) was calculated as:

$${TF}_{1} = \frac{uniq tumor fragments at baseline subs in plasma}{Total fragments at baseline subs in plasma}$$

In the second approach, tumor fraction (TF₂) was calculated from the relationship:

M=N(1−(1−TF₂)^{cov})

where M is the number of tracked sites in a particular sample that have at least one aberrant read, and N is the total number of tracked sites for a patient's monitoring series. TF₁ was more accurate at higher TF levels, and TF₂ was more accurate at lower TF levels.

Background TF and the associated noise/error was estimated by evaluating TF in 48 healthy normal plasma samples, which indicated that TF background in these healthy samples was less than a baseline value of 0.01% TF.

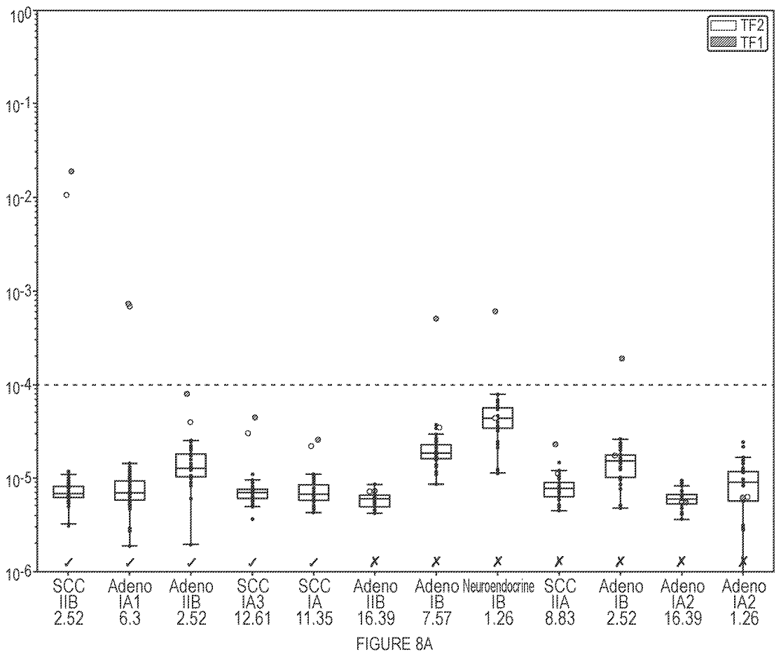

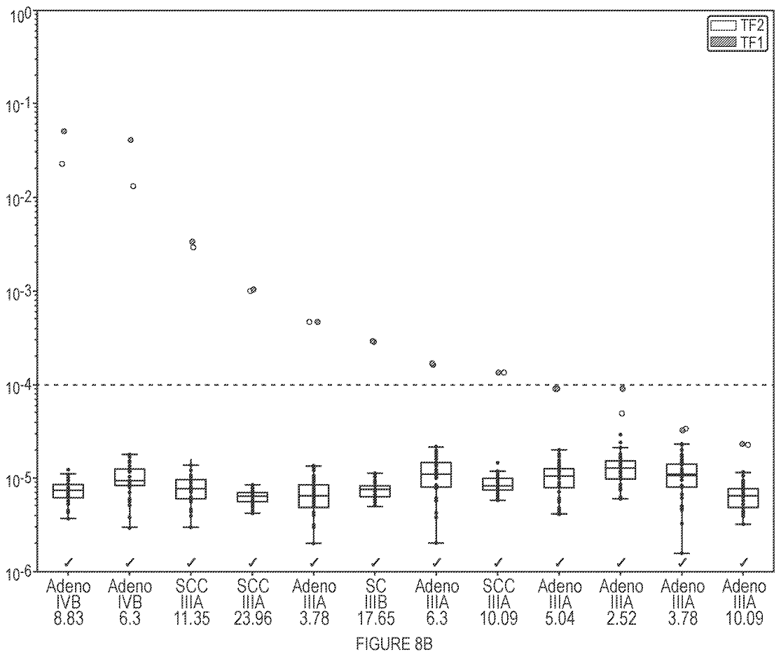

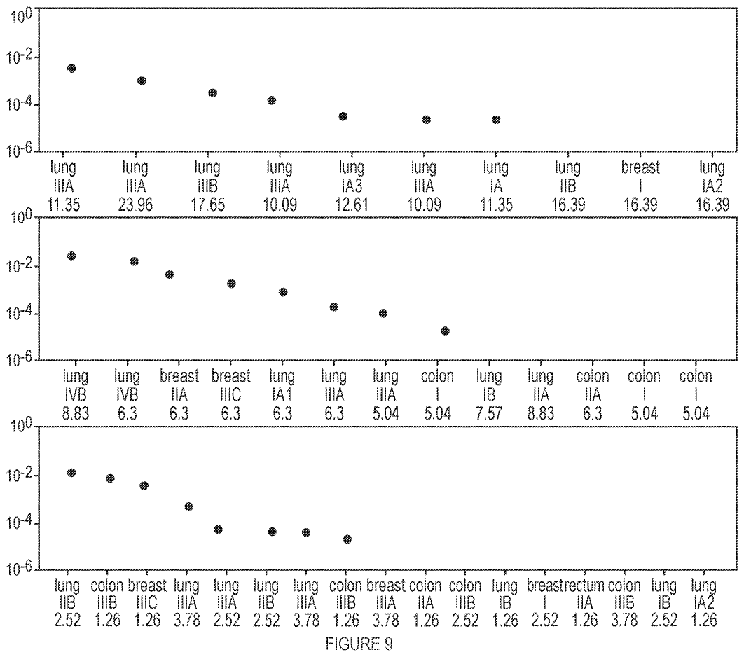

A wide distribution of tumor fractions were observed in a 40 patient subset of 48 affected patients (samples for 8 patients were excluded due to quality control problems with the tumor tissue). The two-step assay workflow could detect tumor fractions as low as 0.01-0.001%. FIGS. 8A-8B provide non-limiting examples of tumor fraction (TF) data (as calculated using the equations for TF₁ and TF₂) for a variety of lung cancer samples determined using the methods disclosed herein. The labels under the X-axis for each figure indicate the NSCLC subtype (upper label), stage (middle label), and tumor mutational burden (TMB) as measured by an in-house genomic profiling assay. FIG. 8A provides a plot of TF (as calculated using the TF₁ and TF₂ approaches, and compared to the background values determined for healthy sample) for plasma samples from early-stage lung cancer patients, for which the overall disease detection rate was 41%. FIG. 8B provides a similar plot of TF data for plasma samples from late-stage lung cancer patients, for which the overall disease detection rate was 100%. Using this approach, the overall disease detection rate was 50% for plasma samples from breast cancer patients, and 30% for plasma samples from colon/rectum cancer patients. As illustrated by the data plotted in FIG. 9, disease detection rates correlated with tumor mutational burden (TMB). The disease detection rate was about 70% for samples exhibiting a TMB greater than 10 (FIG. 9, upper panel). The disease detection rate was about 62% for samples exhibiting a TMB of greater than 5 and less than 10 (FIG. 9, middle panel). The disease detection rate was about 47% for samples exhibiting a TMB of less than or equal to 5.

As determined from the data for paired samples (i.e., paired tissue/buffy coat samples), about 9,000 personalized hypermethylation loci could be tracked per patient based a detected hypermethylation signal, while about 5,000 personalized hypomethylation loci could be tracked per patient based on a detected hypomethylation signal. A higher fraction of the hypomethylation portion of the targeted panel of genomic loci exhibited aberrant methylation.

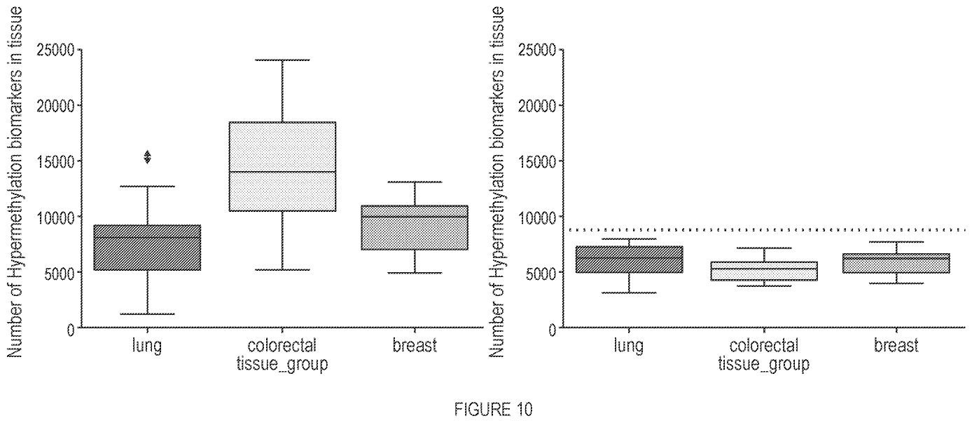

FIG. 10 provides a non-limiting example of data for the distributions of the number of hypermethylated loci (left panel) and hypomethylated loci (right panel) that were observed for three types of cancer, i.e., lung cancer, colorectal cancer, and breast cancer.

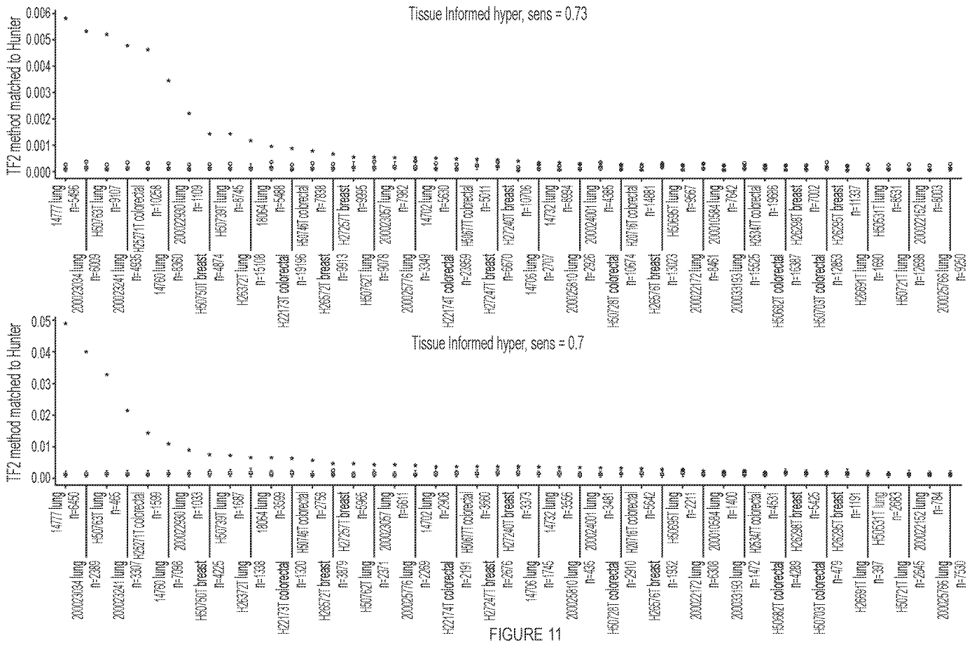

FIG. 11 provides a non-limiting example of data illustrating that tracking personalize methylation biomarkers provides for informed estimates of tumor fraction. The consensus methylation fraction (CCMF) values obtained using the two-step, tissue informed assay were approximately 5× the values obtained using the one-step, tissue naïve assay, while the consensus unmethylation fraction (CCUF) values obtained using the two-step, tissue informed assay were about 1.3× the values obtained using the one-step, tissue naïve assay.

Collectively, the observations from these data indicate that: (i) for plasma samples, the disease-correlated signal is enriched for the genomic locations that are methylated in the tumor, and (ii) for plasma samples, typically most but not all of the hyper false positive (FP) signal (i.e., cancer signal observed in a patient unaffected by cancer) is also present in matched buffy coat samples.

### EXEMPLARY IMPLEMENTATIONS

Exemplary implementations of the methods and systems described herein include:

1. A method comprising:
providing a plurality of nucleic acid molecules obtained from a first sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a first set of sequence read data for a plurality of sequence reads derived from the first sample that represent the captured nucleic acid molecules;
receiving, at one or more processors, the first set of sequence read data for the plurality of sequence reads derived from the first sample from the subject;
determining, using the one or more processors, a methylation status for each of a plurality of genomic loci based on the first set of sequence read data; and
identifying, using the one or more processors, a subset of the plurality of genomic loci that are differentially methylated in the first sample based on a comparison of their methylation status to one or more predetermined methylation status thresholds.

2. The method of clause 1, wherein the subset of differentially methylated genomic loci identified for the subject are used to monitor a disease status of the subject, the method further comprising:
receiving, at the one or more processors, a second set of sequence read data for a plurality of sequence reads derived from a second sample from the subject;
determining, using the one or more processors, a methylation status for the subset of differentially methylated genomic loci based on the second set of sequence read data; and
detecting a disease signal in the subject based on the methylation status determined for the subset of differentially methylated genomic loci in the second set of sequence read data, wherein the disease signal is indicative of the disease status of the subject.

3. The method of clause 2, further comprising estimating a disease-associated DNA fraction for the second sample based on the methylation status determined at the subset of differentially methylated genomic loci in the second set of sequence read data.

4. The method of clause 3, wherein the estimation of disease-associated DNA fraction is based on a fraction of the subset of differentially methylated genomic loci for which methylation is detected, an average level of methylation for the subset of differentially methylated genomic loci, a fraction of differentially methylated sequence reads in the second set of sequence read data, or an absolute number of differentially methylated reads in the second set of sequence read data.

5. The method of any one of clauses 1 to 4, wherein the plurality of genomic loci are identified based on an analysis of methylation status at genomic loci in one or more samples from healthy individuals.

6. The method of any one of clauses 1 to 5, further comprising:
receiving, at the one or more processors, a third set of sequence read data for a plurality of sequence reads derived from a disease-free control sample from the subject;
determining, using the one or more processors, a methylation status for each of the plurality of genomic loci based on the third set of sequence read data; and
excluding genomic loci from the subset of differentially methylated genomic loci if their methylation status is the same for the first sample and the disease-free control sample.

7. The method of any one of clauses 1 to 6, wherein the first sample is a tumor tissue sample.

8. The method of any one of clauses 1 to 7, wherein each set of sequence read data comprises sequence read data derived from a methylation sequencing method.

9. The method of clause 8, wherein the methylation sequencing method comprises use of a bisulfite conversion reaction or an enzymatic conversion reaction to convert non-methylated cytosine to uracil.

10. The method of clause 8, wherein the methylation sequencing method comprises use of a chemical or enzymatic conversion reaction to convert methylated cytosine to uracil.

11. The method of any one of clauses 1 to 7, wherein the subject is suspected of having or is determined to have cancer.

12. The method of clause 11, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

13. The method of clause 11, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

14. The method of any one of clauses 11 to 13, further comprising treating the subject with an anti-cancer therapy.

15. The method of clause 14, wherein the anti-cancer therapy comprises a targeted anti-cancer therapy.

16. The method of clause 15, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), 80elinexor80b8080 sulfate (Vitrakvi), 80elinexor80 mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), 81elinexor81b81 (Gazyva), ofatumumab (Arzerra), 81elinexo (Lynparza), olaratumab (Lartruvo), 81elinexor81b (Tagrisso), 81elinexor81b (Ibrance), panitumumab (Vectibix), 81elinexor81b81 (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), 81elinexor81b govitecan-hziy (Trodelvy), seliciclib, 81elinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

17. The method of any one of clauses 1 to 16, further comprising obtaining the first and/or second sample from the subject.

18. The method of any one of clauses 1 to 17, wherein the first and/or second sample comprises a tissue biopsy sample, a liquid biopsy sample, or a normal control.

19. The method of clause 18, wherein the first and/or second sample is a liquid biopsy sample and comprises blood, plasma, cerebrospinal fluid, sputum, stool, urine, or saliva.

20. The method of clause 19, wherein the first and/or second sample is a liquid biopsy sample and comprises circulating tumor cells (CTCs).

21. The method of clause 18, wherein the first and/or second sample is a liquid biopsy sample and comprises cell-free DNA (cfDNA), circulating tumor DNA (ctDNA), or any combination thereof.

22. The method of any one of clauses 1 to 21, wherein the plurality of nucleic acid molecules comprises a mixture of tumor nucleic acid molecules and non-tumor nucleic acid molecules.

23. The method of clause 20, wherein the tumor nucleic acid molecules are derived from a tumor portion of a heterogeneous tissue biopsy sample, and the non-tumor nucleic acid molecules are derived from a normal portion of the heterogeneous tissue biopsy sample.

24. The method of clause 22, wherein the first and/or second sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

25. The method of any one of clauses 1 to 24, wherein the one or more adapters comprise amplification primers, flow cell adaptor sequences, substrate adapter sequences, or sample index sequences.

26. The method of any one of clauses 1 to 25, wherein the captured nucleic acid molecules are captured from the amplified nucleic acid molecules by hybridization to one or more bait molecules.

27. The method of clause 26, wherein the one or more bait molecules comprise one or more nucleic acid molecules, each comprising a region that is complementary to a region of a captured nucleic acid molecule.

28. The method of any one of clauses 1 to 27, wherein amplifying nucleic acid molecules comprises performing a polymerase chain reaction (PCR) amplification technique, a non-PCR amplification technique, or an isothermal amplification technique.

29. The method of any one of clauses 1 to 28, wherein the sequencing comprises use of a massively parallel sequencing (MPS) technique, whole genome sequencing (WGS), whole exome sequencing, targeted sequencing, direct sequencing, or Sanger sequencing technique.

30. The method of clause 29, wherein the sequencing comprises massively parallel sequencing, and the massively parallel sequencing technique comprises next generation sequencing (NGS).

31. The method of any one of clauses 1 to 30, wherein the sequencer comprises a next generation sequencer.

32. The method of any one of clauses 1 to 31, wherein one or more of the plurality of sequencing reads overlap one or more gene loci within one or more subgenomic intervals in the sample.

33. The method of clause 32, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

34. The method of clause 32 or clause 33, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.

35. The method of clause 32 or clause 33, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1B, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K8, PIGF, PTCH, RAF, RANKL, RET, ROS1, SLAMF7, VEGF, VEGFA, VEGFB, or any combination thereof.

36. The method of any one of clauses 1 to 35, further comprising generating, by the one or more processors, a report indicating the presence or absence of detected disease in the subject.

37. The method of clause 36, further comprising transmitting the report to a healthcare provider.

38. The method of clause 37, wherein the report is transmitted via a computer network or a peer-to-peer connection.

39. A method for identifying a subset of differentially methylated genomic loci in a subject, the method comprising:
a) receiving, at one or more processors, a first set of sequence read data for a plurality of sequence reads derived from a first sample from the subject;
b) determining, using the one or more processors, a methylation status for each of a plurality of genomic loci based on the first set of sequence read data; and
c) identifying, using the one or more processors, a subset of the plurality of genomic loci that are differentially methylated in the first sample based on a comparison of their methylation status to one or more predetermined methylation status thresholds.

40. The method of clause 39, wherein the subset of differentially methylated genomic loci identified for the subject are used to monitor a disease status of the subject, the method further comprising:
d) receiving, at one or more processors, a second set of sequence read data for a plurality of sequence reads derived from a liquid biopsy sample from the subject;
e) determining, using the one or more processors, a methylation status for the subset of differentially methylated genomic loci based on the second set of sequence read data; and
f) detecting a disease signal in the subject based on the methylation status determined for the subset of differentially methylated genomic loci in the second set of sequence read data, wherein the disease signal is indicative of the disease status of the subject.

41. The method of clause 39, further comprising estimating a disease-associated DNA fraction for the liquid biopsy sample based on the methylation status determined at the subset of differentially methylated genomic loci in the second set of sequence read data.

42. The method of clause 41, wherein the estimation of disease-associated DNA fraction is based on a fraction of the subset of differentially methylated genomic loci for which methylation is detected, an average level of methylation for the subset of differentially methylated genomic loci, a fraction of differentially methylated sequence reads in the second set of sequence read data, or an absolute number of differentially methylated reads in the second set of sequence read data.

43. The method of any one of clauses 39 to 42, wherein the plurality of genomic loci are identified based on an analysis of methylation status at genomic loci in one or more samples from healthy individuals.

44. The method of any one of clauses 39 to 43, further comprising:
receiving, at the one or more processors, a third set of sequence read data for a plurality of sequence reads derived from a disease-free control sample from the subject;
determining, using the one or more processors, a methylation status for each of the plurality of genomic loci based on the third set of sequence read data; and
excluding genomic loci from the subset of differentially methylated genomic loci identified in © if their methylation status is the same for the first sample and the disease-free control sample.

45. The method of clause 44, wherein the disease-free control sample comprises a matched normal tissue sample from the subject.

46. The method of clause 44, wherein the disease-free control sample comprises a buffy coat sample from the subject.

47. The method of clause 44, wherein the disease-free control sample comprises an adjacent normal tissue sample from the subject.

48. The method of any one of clauses 40 to 47, wherein the detection of the disease signal is further based on detection of one or more somatic single base substitutions, indels, or structural variants based on the second set of sequence read data.

49. The method of any one of clauses 39 to 48, wherein the plurality of genomic loci comprises at least 10, at least 100, at least 1,000, at least 5,000, at least 10,000, at least 20,000, at least 30,000, at least 40,000, at least 50,000, at least 60,000, at least 70,000, at least 80,000, at least 90,000, at least 100,000, at least 120,000, at last 140,000, at least 160,000, at least 180,000, or at least 200,000 genomic loci.

50. The method of any one of clauses 39 to 49, wherein the identified subset of the plurality of genomic loci is tumor-specific for a subject that has been diagnosed with cancer.

51. The method of any one of clauses 39 to 49, wherein the identified subset of the plurality of genomic loci is treatment-specific for a subject that has previously been treated with an anti-cancer therapy.

52. The method of any one of clauses 39 to 51, wherein the identified subset of the plurality of genomic loci is patient-specific.

53. The method of any one of clauses 39 to 52, wherein the one or more predetermined methylation status thresholds are determined based on an average degree of methylation detected at a specified plurality of genomic loci in a cohort of healthy and diseased subjects.

54. The method of any one of clauses 39 to 52, wherein the one or more predetermined methylation status thresholds are determined based on an analysis of cluster consensus methylation fractions (CCMFs) for a cohort of healthy and diseased subjects.

55. The method of any one of clauses 39 to 52, wherein the one or more predetermined methylation status thresholds are determined based on an analysis of cluster consensus unmethylation fractions (CCUFs) for a cohort of healthy and diseased subjects.

56. The method of clause 54 or clause 55, wherein determining a cluster consensus methylation fraction (CCMF) or a cluster consensus unmethylation fraction (CCUF) for each of the subjects in the cohort of heathy and diseased subjects comprises:
receiving, at one or more processors, sequence read data for a plurality of sequence reads derived from a sample from a subject, wherein at least a portion of the plurality of sequence reads corresponds to a genomic locus comprising a cluster of two or more CpG dinucleotides;
determining, using the one or more processors, a consensus methylation pattern or a consensus unmethylation pattern for the cluster, wherein the consensus methylation pattern represents each CpG dinucleotide in the cluster for which methylation was detected based on the absence cytosine conversion in at least one sequence read from the sequence read data, and wherein the consensus unmethylation pattern represents each CpG dinucleotide in the cluster for which methylation was not detected;
generating, using the one or more processors, a cluster consensus fraction (CCF) for the cluster, wherein the CCF represents a fraction of sequence reads corresponding to the cluster that show the consensus methylation pattern, or a fraction of sequence reads corresponding to the cluster that show the consensus unmethylation pattern, out of a total number of sequence reads from the plurality corresponding to the cluster; and
detecting one or more of the methylation level or the unmethylation level of the cluster based on the CCF.

57. The method of any one of clauses 39 to 56, wherein the one or more predetermined methylation status thresholds are determined for the subject based on a comparison of the methylation status for each of the plurality of genomic loci as determined based on the first set of sequence read data and as determined based on a set of sequence read data derived from a control sample from the subject.

58. The method of clause 57, wherein the control sample comprises a matched normal tissue sample from the subject.

59. The method of any one of clauses 39 to 58, wherein the methylation status of a given genomic locus of the plurality of genomic loci comprises a hypermethylation status, a hypomethylation status, or a hemimethylation status.

60. The method of any one of clauses 39 to 59, wherein the liquid biopsy sample comprises blood, plasma, serum, cerebrospinal fluid, sputum, stool, urine, or saliva.

61. The method of any one of clauses 39 to 60, further comprising performing a sequencing assay to generate the first set of sequence read data, the second set of sequence read data, or both.

62. The method of any one of clause 39 to 61, further comprising performing a sequencing assay to generate the third set of sequence read data.

63. The method of clause 61 or clause 62, wherein the sequencing assay used to generate the first set, second set, or third set of sequence read data comprises a targeted sequencing method.

64. The method of clause 63, wherein the targeted sequencing method comprises use of a set of bait molecules designed to target the subset of differentially methylated genomic loci.

65. The method of clause 64, wherein the set of bait molecules is customized to target a set of differentially methylated genomic loci identified in the subject.

66. The method of clause 61 or clause 62, wherein the sequencing assay used to generate the first set, second set, or third set of sequence read data comprises a whole exome sequencing method.

67. The method of clause 61 or clause 62, wherein the sequencing assay used to generate the first set, second set, or third set of sequence read data comprises a whole genome sequencing assay.

68. The method of any one of clauses 39 to 67, wherein each set of sequence read data comprises sequence read data derived from a methylation sequencing method.

69. The method of clause 68, wherein the methylation sequencing method comprises use of a bisulfite conversion reaction or an enzymatic conversion reaction to convert non-methylated cytosine to uracil.

70. The method of clause 68, wherein the methylation sequencing method comprises use of a chemical or enzymatic conversion reaction to convert methylated cytosine to uracil.

71. The method of clause 68, wherein the methylation sequencing method comprises use of an oxidative bisulfite or enzymatic reaction to detect hydroxymethylation (hmC).

72. The method of any one of clauses 39 to 67, wherein at least one set of sequence read data comprises sequence read data derived from a methylation microarray method.

73. The method of any one of clauses 40 to 72, wherein the disease signal comprises a minimum residual disease (MRD) signal.

74. The method of any one of clauses 40 to 73, wherein the disease signal comprises a tumor response monitoring (TRM) signal.

75. The method of any one of clauses 41 to 73, wherein the disease signal comprises a transplant rejection signal.

76. The method of any one of clauses 39 to 75, wherein the first sample comprises a tumor tissue sample.

77. The method of any one of clauses 41 to 76, wherein the disease-associated DNA comprises circulating tumor DNA (ctDNA).

78. A method for detection of cancer in a subject, the method comprising:
a) receiving, at one or more processors, a first set of sequence read data for a plurality of sequence reads derived from a first sample from the subject;
b) determining, using the one or more processors, a methylation status for each of a plurality of genomic loci based on the first set of sequence read data;
c) identifying, using the one or more processors, a subset of the plurality of genomic loci that are differentially methylated in the first sample based on a comparison of their methylation status to one or more predetermined methylation status thresholds;
d) receiving, at one or more processors, methylation status data for the subset of differentially methylated genomic loci in a liquid biopsy sample from the subject; and
e) detecting, using the one or more processors, a presence of the cancer in the subset based on the methylation status data for the subset of differentially methylated genomic loci in the liquid biopsy sample.

79. The method of clause 78, wherein the methylation status data for the subset of differentially methylated genomic loci is generated using a PCR-based detection assay, a quantitative PCR-based detection assay, a digital droplet PCR-based detection assay, a methylation-specific PCR-based detection assay, or a microarray-based DNA methylation profiling assay.

80. The method of clause 78 or clause 79, further comprising estimating a circulating tumor DNA fraction for the first sample and/or the liquid biopsy sample based on the methylation status data for the subset of differentially methylated genomic loci.

81. The method of clause 80, wherein the estimation of circulating tumor DNA fraction is based on a fraction of the subset of differentially methylated genomic loci for which methylation is detected, or an average level of methylation for the subset of differentially methylated genomic loci.

82. The method of any one of clauses 78 to 81, further comprising:
receiving, at the one or more processors, a second set of sequence read data for a plurality of sequence reads derived from a control sample from the subject;
determining, using the one or more processors, a methylation status for each of the plurality of genomic loci based on the second set of sequence read data; and
excluding genomic loci from the subset of differentially methylated genomic loci identified in step 74© based on a comparison of their methylation status determined based on the second set of sequencing data to the one or more predetermined methylation status thresholds.

83. The method of clause 82, wherein the control sample comprises a matched normal tissue sample from the subject.

84. The method of clause 82, wherein the control sample comprises a buffy coat sample from the subject.

85. The method of any one of clauses 78 to 84, wherein the detection of the presence of cancer in the subject further comprises detection of one or more somatic single base substitutions, indels, or structural variants based on the first set of sequence read data or an analysis of the liquid biopsy sample.

86. The method of any one of clauses 78 to 85, wherein the plurality of genomic loci comprises at least 10, at least 100, at least 1,000, at least 5,000, at least 10,000, at least 20,000, at least 30,000, at least 40,000, at least 50,000, at least 60,000, at least 70,000, at least 80,000, at least 90,000, at least 100,000, at least 120,000, at last 140,000, at least 160,000, at least 180,000, or at least 200,000 genomic loci.

87. The method of any one of clauses 78 to 86, wherein the identified subset of the plurality of genomic loci is tumor-specific for a subject that has been diagnosed with cancer.

88. The method of any one of clauses 78 to 87, wherein the identified subset of the plurality of genomic loci is treatment-specific for a subject that has previously been treated with an anti-cancer therapy.

89. The method of any one of clauses 78 to 88, wherein the identified subset of the plurality of genomic loci is patient-specific.

90. The method of any one of clauses 78 to 89, wherein the one or more predetermined methylation status thresholds are determined based on an average degree of methylation detected at a specified plurality of genomic loci in a cohort of healthy and diseased subjects.

91. The method of any one of clauses 78 to 90, wherein the one or more predetermined methylation status thresholds are determined based on an analysis of cluster consensus methylation fractions (CCMFs) for a cohort of healthy and diseased subjects.

92. The method of any one of clauses 78 to 91, wherein the one or more predetermined methylation status thresholds are determined based on an analysis of cluster consensus unmethylation fractions (CCUFs) for a cohort of healthy and diseased subjects.

93. The method of clause 91 or clause 92, wherein determining a cluster consensus methylation fraction (CCMF) or a cluster consensus unmethylation fraction (CCUF) for each of the subjects in the cohort of heathy and diseased subjects comprises:
receiving, at one or more processors, sequence read data for a plurality of sequence reads derived from a sample from a subject, wherein at least a portion of the plurality of sequence reads corresponds to a genomic locus comprising a cluster of two or more CpG dinucleotides;
determining, using the one or more processors, a consensus methylation pattern or a consensus unmethylation pattern for the cluster, wherein the consensus methylation pattern represents each CpG dinucleotide in the cluster for which methylation was detected based on the absence cytosine conversion in at least one sequence read from the sequence read data, and wherein the consensus unmethylation pattern represents each CpG dinucleotide in the cluster for which methylation was not detected;
generating, using the one or more processors, a cluster consensus fraction (CCF) for the cluster, wherein the CCF represents a fraction of sequence reads corresponding to the cluster that show the consensus methylation pattern, or a fraction of sequence reads corresponding to the cluster that show the consensus unmethylation pattern, out of a total number of sequence reads from the plurality corresponding to the cluster; and detecting one or more of the methylation level or the unmethylation level of the cluster based on the CCF.

94. The method of any one of clauses 78 to 93, wherein the one or more predetermined methylation status thresholds are determined for the subject based on a comparison of the methylation status for each of the plurality of genomic loci as determined based on the first set of sequence read data and as determined based on a set of sequence read data derived from a control sample from the subject.

95. The method of clause 94, wherein the control sample a matched normal tissue sample from the subject.

96. The method of any one of clauses 78 to 95, wherein the methylation status of a given genomic locus of the plurality of genomic loci comprises a hypermethylation status, a hypomethylation status, or a hemimethylation status.

97. The method of any one of clauses 78 to 96, wherein the liquid biopsy sample comprises blood, plasma, serum, cerebrospinal fluid, sputum, stool, urine, or saliva.

98. The method of any one of clauses 78 to 97, further comprising performing a sequencing assay to generate the first set of sequence read data.

99. The method of clause 98, wherein the sequencing assay used to generate the first set of sequence read data comprises a targeted sequencing method.

100. The method of clause 99, wherein the targeted sequencing method comprises use of a set of bait molecules designed to target the subset of differentially methylated genomic loci.

101. The method of clause 100, wherein the set of bait molecules is customized to target a set of differentially methylated genomic loci identified in the subject.

102. The method of any one of clauses 78 to 101, wherein the methylation status data for the subset of differentially methylated genomic loci is generated using a PCR-based detection assay, a quantitative PCR-based detection assay, a digital droplet PCR-based detection assay, or a methylation-specific PCR-based detection assay and a custom set of PCR primers designed to target a set of differentially methylated genomic loci identified in the subject.

103. A method for diagnosing a disease, the method comprising:
diagnosing that a subject has the disease based on a determination of personalized methylation biomarkers for a sample from the subject, wherein the personalized methylation biomarkers are determined according to the method of any one of clauses 39 to 102.

104. A method of selecting an anti-cancer therapy, the method comprising:
responsive to determining personalized methylation biomarkers for a sample from a subject, selecting an anti-cancer therapy for the subject, wherein the personalized methylation biomarkers are determined according to the method of any one of clauses 39 to 102.

105. A method of treating a cancer in a subject, comprising:
responsive to determining personalized methylation biomarkers for a sample from the subject, administering an effective amount of an anti-cancer therapy to the subject, wherein the personalized methylation biomarkers is determined according to the method of any one of clauses 39 to 102.

106. A method for monitoring cancer progression or recurrence in a subject, the method comprising:
detecting a first disease signal corresponding to cancer based on a methylation status determined for a subset of differentially methylated genomic loci identified in sequence read data derived from a sample obtained from the subject at a first time point, where the disease signal is detected according to the method of any one of clauses 39 to 102;
detecting a second disease signal corresponding to cancer based on a methylation status determined for a subset of differentially methylated genomic loci identified in sequence read data derived from a sample obtained from the subject at a second time point; and comparing the first disease signal to the second disease signal, thereby monitoring the cancer progression or recurrence.

107. The method of clause 106, wherein the second disease signal is detected according to the method of any one of clauses 39 to 102.

108. The method of clause 106 or clause 107, further comprising selecting an anti-cancer therapy for the subject in response to the cancer progression.

109. The method of clause 106 or clause 107, further comprising administering an anti-cancer therapy to the subject in response to the cancer progression.

110. The method of clause 106 or clause 107, further comprising adjusting an anti-cancer therapy for the subject in response to the cancer progression.

111. The method of any one of clauses 108 to 110, further comprising adjusting a dosage of the anti-cancer therapy or selecting a different anti-cancer therapy in response to the cancer progression.

112. The method of clause 111, further comprising administering the adjusted anti-cancer therapy to the subject.

113. The method of any one of clauses 106 to 112, wherein the first time point is before the subject has been administered an anti-cancer therapy, and wherein the second time point is after the subject has been administered the anti-cancer therapy.

114. The method of any one of clauses 106 to 113, wherein the subject has a cancer, is at risk of having a cancer, is being routine tested for cancer, or is suspected of having a cancer.

115. The method of any one of clauses 106 to 114, wherein the cancer is a solid tumor.

116. The method of any one of clauses 106 to 114, wherein the cancer is a hematological cancer.

117. The method of any one of clauses 108 to 116, wherein the anti-cancer therapy comprises chemotherapy, radiation therapy, immunotherapy, a targeted therapy, or surgery.

118. The method of any one of clauses 39 to 102, further comprising determining, identifying, or applying a methylation status value determined for the subset of differentially methylated genomic loci as a diagnostic value associated with the sample.

119. The method of any one of clauses 39 to 102, further comprising generating a genomic profile for the subject based on a determination of personalized methylation biomarkers.

120. The method of clause 119, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.

121. The method of clause 119 or clause 120, wherein the genomic profile for the subject further comprises results from a nucleic acid sequencing-based test.

122. The method of any one of clauses 119 to 121, further comprising selecting an anti-cancer therapy, administering an anti-cancer therapy, or applying an anti-cancer therapy to the subject based on the generated genomic profile.

123. The method of any one of clauses 40 to 102, wherein the detection of the disease signal is used in making suggested treatment decisions for the subject.

124. The method of any one of clauses 40 to 102, wherein the detection of the disease signal is used in applying or administering a treatment to the subject.

125. A system comprising:
one or more processors; and
a memory communicatively coupled to the one or more processors and configured to store instructions that, when executed by the one or more processors, cause the system to:
receive a first set of sequence read data for a plurality of sequence reads derived from a first sample from the subject;
determine a methylation status for each of a plurality of genomic loci based on the first set of sequence read data; and
identify a subset of the plurality of genomic loci that are differentially methylated in the first sample based on a comparison of their methylation status to one or more predetermined methylation status thresholds.

126. The system of clause 125, further comprising instructions that, when executed by the one or more processors, cause the system to:
receive a second set of sequence read data for a plurality of sequence reads derived from a liquid biopsy sample from the subject;
determine a methylation status for the subset of differentially methylated genomic loci based on the second set of sequence read data; and
detect a disease signal in the subject based on the methylation status determined for the subset of differentially methylated genomic loci in the second set of sequence read data, wherein the disease signal is indicative of the disease status of the subject.

127. A non-transitory computer-readable storage medium storing one or more programs, the one or more programs comprising instructions, which when executed by one or more processors of a system, cause the system to:
receive a first set of sequence read data for a plurality of sequence reads derived from a first sample from the subject;
determine a methylation status for each of a plurality of genomic loci based on the first set of sequence read data; and
identify a subset of the plurality of genomic loci that are differentially methylated in the first sample based on a comparison of their methylation status to one or more predetermined methylation status thresholds.

128. The non-transitory computer-readable storage medium of clause 127, further comprising instructions that, when executed by the one or more processors of a system, cause the system to:
receive a second set of sequence read data for a plurality of sequence reads derived from a liquid biopsy sample from the subject;
determine a methylation status for the subset of differentially methylated genomic loci based on the second set of sequence read data; and
detect a disease signal in the subject based on the methylation status determined for the subset of differentially methylated genomic loci in the second set of sequence read data, wherein the disease signal is indicative of the disease status of the subject.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method comprising:
providing a plurality of nucleic acid molecules obtained from a first sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a first set of sequence read data for a plurality of sequence reads derived from the first sample that represent the captured nucleic acid molecules;
receiving, at one or more processors, the first set of sequence read data for the plurality of sequence reads derived from the first sample from the subject;
determining, using the one or more processors, a methylation status for each of a plurality of genomic loci based on the first set of sequence read data; and
identifying, using the one or more processors, a subset of the plurality of genomic loci that are differentially methylated in the first sample based on a comparison of their methylation status to one or more predetermined methylation status thresholds.

2. The method of claim 1, wherein the subset of differentially methylated genomic loci identified for the subject are used to monitor a disease status of the subject, the method further comprising:
receiving, at one or more processors, a second set of sequence read data for a plurality of sequence reads derived from a liquid biopsy sample from the subject;
determining, using the one or more processors, a methylation status for the subset of differentially methylated genomic loci based on the second set of sequence read data; and
detecting a disease signal in the subject based on the methylation status determined for the subset of differentially methylated genomic loci in the second set of sequence read data, wherein the disease signal is indicative of the disease status of the subject.

3. The method of claim 1, further comprising estimating a circulating tumor DNA (ctDNA) fraction for the liquid biopsy sample based on the methylation status determined at the subset of differentially methylated genomic loci in the second set of sequence read data.

4. (canceled)

5. The method of claim 1, wherein the plurality of genomic loci are identified based on an analysis of methylation status at genomic loci in one or more samples from healthy individuals.

6. The method of claim 1, further comprising:
receiving, at the one or more processors, a third set of sequence read data for a plurality of sequence reads derived from a disease-free control sample from the subject;
determining, using the one or more processors, a methylation status for each of the plurality of genomic loci based on the third set of sequence read data; and
excluding genomic loci from the identified subset of differentially methylated genomic loci if their methylation status is the same for the first sample and the disease-free control sample.

7. (canceled)

8. The method of claim 2, wherein the detection of the disease signal is further based on detection of one or more somatic single base substitutions, indels, or structural variants based on the second set of sequence read data.

9. The method of claim 1, wherein the identified subset of the plurality of genomic loci is tumor-specific for a subject that has been diagnosed with cancer.

10. The method of claim 1, wherein the identified subset of the plurality of genomic loci is treatment-specific for a subject that has previously been treated with an anti-cancer therapy.

11. The method of claim 1, wherein the identified subset of the plurality of genomic loci is patient-specific.

12. The method of claim 1, wherein the one or more predetermined methylation status thresholds are determined based on: (i) an average degree of methylation detected at a specified plurality of genomic loci, (ii) an analysis of cluster consensus methylation fractions (CCMFs), or (iii) an analysis of cluster consensus unmethylation fractions (CCUFs) in a cohort of healthy and diseased subjects.

13. (canceled)

14. (canceled)

15. The method of claim 1, wherein the one or more predetermined methylation status thresholds are determined for the subject based on a comparison of the methylation status for each of the plurality of genomic loci as determined based on the first set of sequence read data and as determined based on a set of sequence read data derived from a control sample from the subject.

16. (canceled)

17. The method of claim 1, wherein each set of sequence read data comprises sequence read data derived from a methylation sequencing method.

18. The method of claim 1, wherein the first sample comprises a tumor tissue sample.

19. A method for detection of cancer in a subject, the method comprising:
a) receiving, at one or more processors, a first set of sequence read data for a plurality of sequence reads derived from a first sample from the subject;
b) determining, using the one or more processors, a methylation status for each of a plurality of genomic loci based on the first set of sequence read data;
c) identifying, using the one or more processors, a subset of the plurality of genomic loci that are differentially methylated in the first sample based on a comparison of their methylation status to one or more predetermined methylation status thresholds;
d) receiving, at one or more processors, methylation status data for the subset of differentially methylated genomic loci in a liquid biopsy sample from the subject; and
e) detecting, using the one or more processors, a presence of the cancer in the subset based on the methylation status data for the subset of differentially methylated genomic loci in the liquid biopsy sample.

20. The method of claim 19, wherein the methylation status data for the subset of differentially methylated genomic loci is generated using a PCR-based detection assay, a quantitative PCR-based detection assay, a digital droplet PCR-based detection assay, a methylation-specific PCR-based detection assay, or a microarray-based DNA methylation profiling assay.

21. The method of claim 19, further comprising estimating a circulating tumor DNA fraction for the first sample and/or the liquid biopsy sample based on the methylation status data for the subset of differentially methylated genomic loci.

22. (canceled)

23. The method of claim 19, further comprising:
receiving, at the one or more processors, a second set of sequence read data for a plurality of sequence reads derived from a control sample from the subject;
determining, using the one or more processors, a methylation status for each of the plurality of genomic loci based on the second set of sequence read data; and
excluding genomic loci from the identified subset of differentially methylated genomic loci based on a comparison of their methylation status determined based on the second set of sequencing data to the one or more predetermined methylation status thresholds.

24. The method of claim 19, wherein the detection of the presence of cancer in the subject further comprises detection of one or more somatic single base substitutions, indels, or structural variants based on the first set of sequence read data or an analysis of the liquid biopsy sample.

25. The method of claim 19, wherein the identified subset of the plurality of genomic loci is tumor-specific, treatment-specific, or patient-specific for a subject that has been diagnosed with cancer.

26. The method of claim 19, wherein the one or more predetermined methylation status thresholds are determined based on: (i) an average degree of methylation detected at a specified plurality of genomic loci, (ii) an analysis of cluster consensus methylation fractions (CCMFs), or (iii) an analysis of cluster consensus unmethylation fractions (CCUFs) in a cohort of healthy and diseased subjects.

27. (canceled)

28. (canceled)

29. The method of claim 19, wherein the one or more predetermined methylation status thresholds are determined for the subject based on a comparison of the methylation status for each of the plurality of genomic loci as determined based on the first set of sequence read data and as determined based on a set of sequence read data derived from a control sample from the subject.

30. A system comprising:
one or more processors; and
a memory communicatively coupled to the one or more processors and configured to store instructions that, when executed by the one or more processors, cause the system to perform a method comprising:
a) receiving a first set of sequence read data for a plurality of sequence reads derived from a first sample from the subject;
b) determining a methylation status for each of a plurality of genomic loci based on the first set of sequence read data; and
c) identifying a subset of the plurality of genomic loci that are differentially methylated in the first sample based on a comparison of their methylation status to one or more predetermined methylation status thresholds.
